(19) United States
(12) Patent Application Publication
(10) Pub. No.: US 2024/0269125 A1
(43) Pub. Date: Aug. 15, 2024

(54) METHODS OF TREATING CORONAVIRUS INFECTIONS BY CO-ADMINISTERING AN FKBP LIGAND AND AN ANTIVIRAL AGENT

(71) Applicant: Tutela Pharmaceuticals, Inc., Vernon Hills, IL (US)

(72) Inventor: William Fitzsimmons, Vernon Hills, IL (US)

(21) Appl. No.: 18/167,843

(22) PCT Filed: Aug. 11, 2021
(86) PCT No.: PCT/US2021/045455

§ 371 Date: Feb. 11, 2023

Related U.S. Application Data
(60) Provisional application No. 63/064,149, filed on Aug. 11, 2020.
(60) Provisional application No. 63/153,521, filed on Feb. 25, 2021.
(60) Provisional application No. 63/186,689, filed on May 10, 2021.

(51) Int. Cl. A61K 31/453; A61K 31/401; A61K 31/4439; A61K 31/444; A61K 31/4995; A61K 31/5025; A61K 45/06; A61P 31/14

(52) U.S. Cl. CPC A61K 31/453; A61K 31/401; A61K 31/4439; A61K 31/444; A61K 31/4995; A61K 31/5025; A61K 45/06; A61P 31/14

(57) ABSTRACT

The present disclosure provides compositions comprising an FKBP ligand or derivative thereof and an antiviral agent such as an RNA polymerase inhibitor and/or a protease inhibitor, and methods of treating or preventing a coronavirus infection comprising co-administering to a subject an FKBP ligand or derivative thereof (optionally at a sub-therapeutic dose) and an antiviral agent such as an RNA polymerase inhibitor and/or a protease inhibitor (optionally at a sub-therapeutic dose).

### PRIORITY CLAIMS

This application claims the benefit of U.S. Provisional Patent Application Ser. No. 63/064,149, filed Aug. 11, 2020; U.S. Provisional Patent Application Ser. No. 63/153,521, filed Feb. 25, 2021; and U.S. Provisional Patent Application Ser. No. 63/186,689, filed May 10, 2021, the entire contents of each of which are relied upon and incorporated herein by reference.

### BACKGROUND

FKBP is one of the naturally occurring ubiquitous intracellular proteins called immunophilins that has enzymatic activity as a peptidyl prolyl cis-trans isomerase and is also essential to the pharmacologic activity of the immunosuppressant tacrolimus by binding to the drug and subsequently inhibiting calcineurin phosphatase. Additionally FKBP is a key intracellular binding protein of the mammalian target of rapamycin (mTOR) inhibitors sirolimus and everolimus. These mTOR inhibitors also are immunosuppressants but do not inhibit calcineurin phosphatase.

The binding of these immunosuppressants, tacrolimus, everolimus and sirolimus, to FKBP is necessary but not sufficient to produce immunosuppression.

However, tacrolimus, everolimus, and sirolimus are potent immunosuppressants with well-known side effect profiles, including an increased risk of some viral infections. Moreover, the mechanism by which tacrolimus produces immunosuppression (calcineurin phosphatase inhibition) also mediates some of the key toxicities (e.g. nephrotoxicity, neurotoxicity, diabetogenicity). For at least these reasons, using FKBP ligands-like tacrolimus, everolimus, or sirolimus—to treat coronavirus infections would present serious and potentially life-threatening risks, especially in high-risk populations like elderly individuals with pre-existing co-morbidities.

A need persists for compositions for treating coronavirus infections that do not substantially suppress a subject's immune response.

### SUMMARY

The present disclosure provides compositions comprising one or more FKBP ligands or derivative thereof, wherein the compositions do not substantially suppress a subject's immune system or response.

In some embodiments, the present disclosure provides a method of treating or preventing a coronavirus infection in a subject in need thereof, the method comprising, consisting essentially of, or consisting of: co-administering to the subject in need thereof an FKBP ligand or derivative thereof and an antiviral agent, optionally wherein at least the subject is administered a sub-therapeutic amount of at least one of the FKBP ligand or derivative thereof and the antiviral agent.

In other embodiments, the present disclosure provides a pharmaceutical composition comprising, consisting essentially of, or consisting of: an FKBP ligand or a derivative thereof; and an antiviral agent, optionally wherein one or both of the FKBP ligand or derivative thereof and the antiviral agent are present in a sub-therapeutic amount.

These and other embodiments are described in further detail herein below.

The detailed description and examples provided herewith depict various embodiments of this disclosure for purposes of illustration only. One skilled in the art will readily recognize from the following discussion that alternative embodiments of the structures and methods illustrated herein may be employed without departing from the principles of embodiments described herein.

### BRIEF DESCRIPTION OF THE FIGURES

FIGS. 1A-1B show graphical representations of in vitro reduction in SARS COV-2 viral CPE and cell control for FK1706 as a function of concentration.

FIG. 2 shows a graphical representation of in vitro viral CPE reduction in SARS COV-2 viral CPE and cell control for a combination of a sub-therapeutic amount of FK1706 and a sub-therapeutic amount of remdesivir (3 μM).

FIG. 3 shows a graphical representation of in vitro viral CPE reduction in SARS COV-2 viral CPE and cell control for remdesivir alone at 3 μM.

FIG. 4 shows a graphical representation of in vitro viral CPE reduction in SARS COV-2 viral CPE and cell control for remdesivir alone across a concentration range of 0.1-100 μM.

FIG. 5 shows a graphical representation of in vitro viral CPE reduction in SARS COV-2 viral CPE and cell control for a combination of a sub-therapeutic amount of FK1706 and a sub-therapeutic amount of M128533 (1 μg/mL).

FIG. 6 shows a graphical representation of in vitro viral CPE reduction in SARS COV-2 viral CPE and cell control for sub-therapeutic M128533 alone at 1 μg/mL.

FIG. 7 shows a graphical representation of in vitro viral CPE reduction in SARS COV-2 viral CPE and cell control for sub-therapeutic M128533 alone across a concentration range of 0.1-100 μg/mL.

FIG. 8 shows a graphical representation of in vitro reduction in SARS COV-2 viral CPE and cell control for ElteN378 alone, as a function of ElteN378 concentration.

FIG. 9 shows a graphical representation of in vitro viral CPE reduction in SARS COV-2 viral CPE and cell control for a combination of ElteN378 and sub-therapeutic remdesivir (3 μM).

FIG. 10 shows a graphical representation of in vitro anti-SARS COV-2 activity of sub-therapeutic remdesivir (3 μM) alone.

FIG. 11 shows a graphical representation of in vitro anti-SARS COV-2 activity of remdesivir at concentrations from 0.1 to 100 μM.

FIG. 12 shows a graphical representation of in vitro anti-SARS COV-2 activity for coadministration ElteN378 and sub-therapeutic M128533 (1 μg/mL).

FIG. 13 shows a graphical representation of in vitro anti-SARS COV-2 activity for sub-therapeutic M128533 (1 μg/ml) alone.

FIG. 14 shows a graphical representation of in vitro anti-SARS COV-2 activity for M128533 monotherapy as a function of concentration from 0.1 to 100 μg/ml.

FIG. 15 shows a graphical representation of in vitro reduction in SARS COV-2 viral CPE (solid circles) and cell control (open circles) for FK1706 alone as a function of FK1706 concentration.

FIG. 16 shows a graphical representation of in vitro reduction in SARS COV-2 activity when FK1706 (2.85-90 UM) is co-administered with sub-therapeutic molnupiravir (0.3 μM).

FIG. 17 shows a graphical representation of anti-SARS COV-2 activity for sub-therapeutic molnupiravir (0.3 μM) monotherapy.

FIG. 18 shows a graphical representation of in vitro reduction in SARS COV-2 viral CPE (solid circles) and cell control (open circles) for GPI-1485 alone as a function of GPI-1485 concentration.

FIG. 19 shows a graphical representation of in vitro reduction in SARS COV-2 activity when GPI-1485 (40-320 μM) is co-administered with sub-therapeutic molnupiravir (0.3 μM).

FIG. 20 shows a graphical representation of in vitro reduction in SARS COV-2 activity when GPI-1485 (40-320 μM) is co-administered with sub-therapeutic remdesivir (3 μM).

FIG. 21 shows a graphical representation of anti-SARS COV-2 activity for sub-therapeutic molnupiravir (0.3 μM) monotherapy.

FIG. 22 shows a graphical representation of anti-SARS COV-2 activity for sub-therapeutic remdesivir (3 μM) monotherapy.

FIG. 23 shows results of in vitro reduction in SARS COV-2 activity when FK1706 (0-60,000 nM) is co-administered with remdesivir (0-6,666.667 nM) in a Vero E6 cell line with a live SARS-CoV-2 virus assay measuring the ability of compounds to reverse the viral induced cytopathic effect (CPE).

FIG. 24 shows single-agent CPE dose response curves for remdesivir (solid circles), molnupiravir (solid squares), and FKBP ligand FK1706 (solid triangles) in a Vero E6 cell SARS-CoV-2 assay.

FIG. 25 shows single-agent cytotoxicity dose response curves for remdesivir (solid circles), molnupiravir (solid squares), and FKBP ligand FK1706 (solid triangles) in a Vero E6 cell SARS-CoV-2 assay.

FIG. 26 shows CPE dose response curves for combination therapy consisting of FKBP ligand FK1706 and remdesivir at various FK1706 concentrations in a Vero E6 cell SARS-COV-2 assay.

FIG. 27 shows cytotoxicity dose response curves for combination therapy consisting of FKBP ligand FK1706 and remdesivir at various FK1706 concentrations in a Vero E6 cell SARS-COV-2 assay.

FIG. 28 shows CPE dose response curves for combination therapy consisting of FKBP ligand FK1706 and remdesivir at various remdesivir concentrations in a Vero E6 cell SARS-COV-2 assay.

FIG. 29 shows cytotoxicity dose response curves for combination therapy consisting of FKBP ligand FK1706 and remdesivir at various remdesivir concentrations in a Vero E6 cell SARS-CoV-2 assay.

FIG. 30 is the WHO Clinical Progression Scale for COVID-19 research studies as published by the WHO Working Group on the Clinical Characterization and Management of COVID-19 Infection.

### DETAILED DESCRIPTION

The present disclosure provides compositions (e.g., pharmaceutical compositions) comprising an FKBP ligand or derivative thereof and methods of treating a coronavirus infection in a subject using same.

### FKBP Ligands and Derivatives Thereof

Compositions of the present disclosure comprise an FKBP ligand or a derivative thereof. As used herein, the term “derivative thereof” refers to non-toxic functional equivalents of a known FKBP ligand, which can be obtained by substitution of atoms or molecular groups or bonds of an FKBP ligand's molecular structure, whereby the basic structure is not changed, and which differ from the structure of an FKBP ligand in at least one position; and includes, without limitation, pharmaceutically acceptable salts, prodrugs, biologically active metabolites, stereoisomers, and isomers of an FKBP ligand that inhibit the replication of coronaviruses.

In some embodiments, the FKBP ligand is tacrolimus. Tacrolimus, also referred to as Prograf, Protopic, FK-506, or fujimycin, inhibits calcineurin and has the following chemical structure:

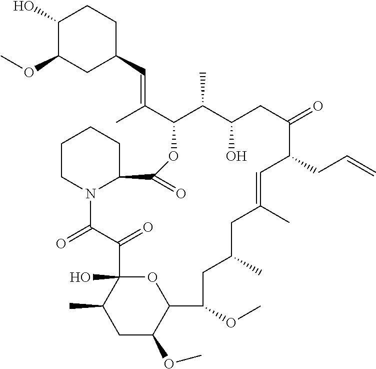 [SMILES: C=CC[C@@H]1/C=C(\C)C[C@H](C)C[C@H](OC)C2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

In some embodiments, the FKBP ligand is everolimus. Everolimus, also referred to as Afinitor and Zortress, inhibits mammalian target of rapamycin (mTOR) and has the following chemical structure:

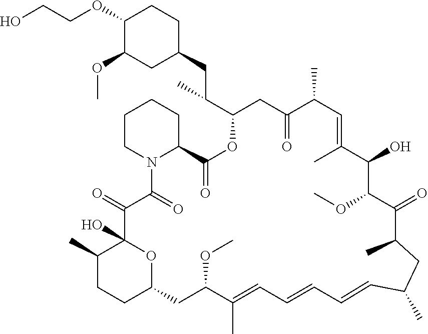 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

In some embodiments, the FKBP ligand is sirolimus. Sirolimus, also referred to as rapamycin, inhibits mTOR and has the following chemical structure:

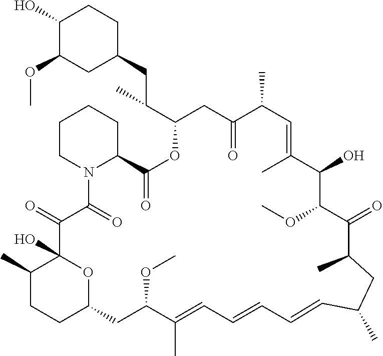 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

In some embodiments, the FKBP ligand is 15-O-demethyl tacrolimus (15-DMT). 15-DMT binds to FKBP and has the following chemical structure:

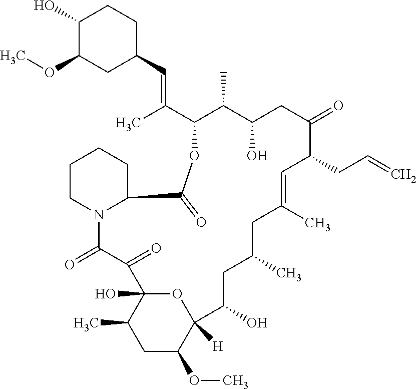 [SMILES: C=CC[C@@H]1/C=C(\C)C[C@H](C)C[C@H](O)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

In some embodiments, the FKBP ligand is GPI1046. GPI1046 binds to FKBP and has the following chemical structure:

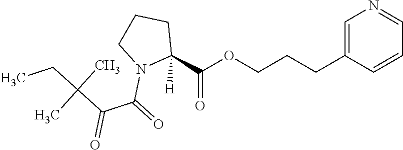 [SMILES: CCC(C)(C)C(=O)C(=O)N1CCC[C@H]1C(=O)OCCCc1cccnc1]

In some embodiments, the FKBP ligand is GPI-1485. GPI-1485 binds to FKBP and has the following chemical structure:

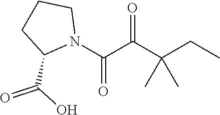 [SMILES: CCC(C)(C)C(=O)C(=O)N1CCC[C@H]1C(=O)O]

In some embodiments, the FKBP ligand is ElteN378. EliteN378 binds to FKBP and has the following chemical structure:

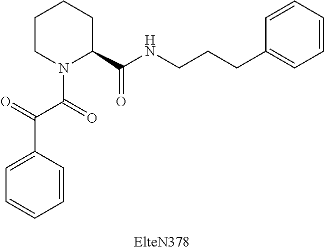 [SMILES: O=C(C(=O)N1CCCC[C@H]1C(=O)NCCCc1ccccc1)c1ccccc1]

In some embodiments, the FKBP ligand is V10367. V10367 binds to FKBP and has the following chemical structure:

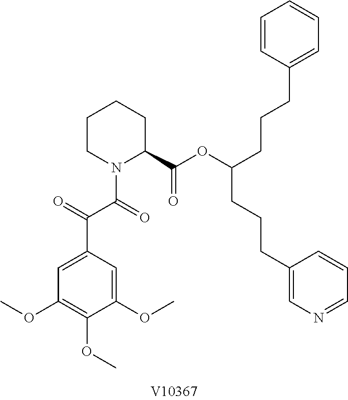 [SMILES: COc1cc(C(=O)C(=O)N2CCCC[C@H]2C(=O)OC(CCCc2ccccc2)CCCc2cccnc2)cc(OC)c1OC]

In some embodiments, the FKBP ligand is L-685818. L-685818 is the C18-hydroxylated, C21-ethyl substituted analog of FK506, binds to FKBP and has the following chemical structure:

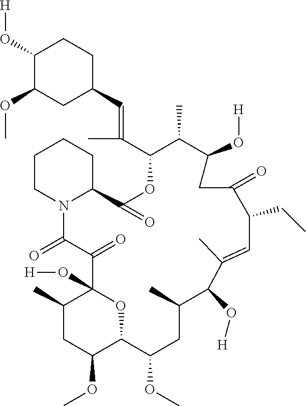 [SMILES: CC[C@@H]1/C=C(\C)[C@@H](O)[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

In some embodiments, the FKBP ligand is WAY-124,466. WAY-124,466 binds with FKBP and has the following chemical structure:

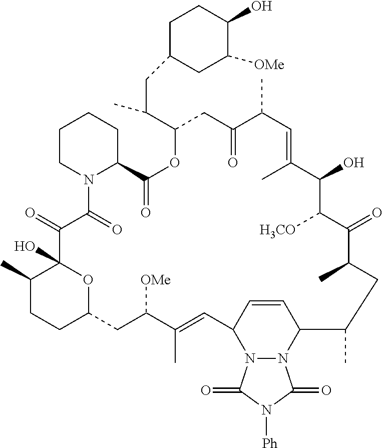

In some embodiments, the FKBP ligand is AG5473. AG5473 binds FKBP and has the following chemical structure:

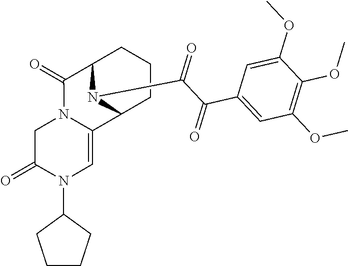 [SMILES: COc1cc(C(=O)C(=O)N2[C@@H]3CCC[C@H]2C(=O)N2CC(=O)N(C4CCCC4)C=C32)cc(OC)c1OC]

In some embodiments, the FKBP ligand is AG5507. AG5507 binds FKBP and has the following chemical structure:

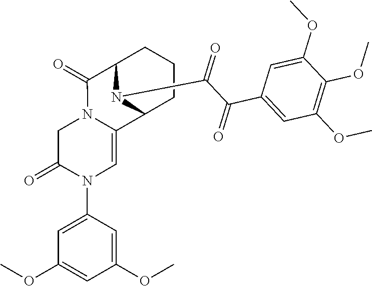

In some embodiments, the FKBP ligand is meridamycin. Meridamycin binds FKBP and has the following chemical structure:

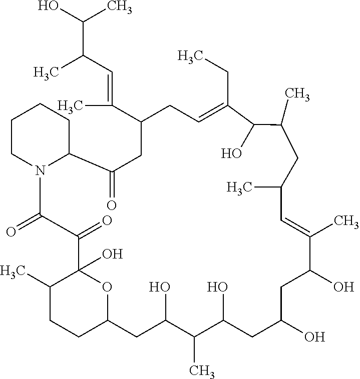 [SMILES: CC/C1=C\CC(/C(C)=C/C(C)C(C)O)CC(=O)C2CCCCN2C(=O)C(=O)C2(O)OC(CCC2C)CC(O)C(C)C(O)CC(O)CC(O)/C(C)=C/C(C)CC(C)C1O]

In some embodiments, the FKBP ligand is FK1706. FK1706 binds FKBP and has the following chemical structure:

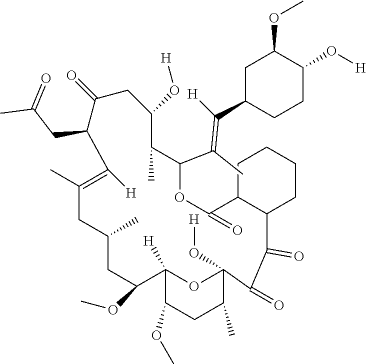

### Pharmaceutical Compositions

The present disclosure provides compositions, including pharmaceutical compositions, comprising an FKBP ligand or derivative thereof.

In some embodiments, the composition includes a pharmaceutically effective amount of the FKBP ligand or derivative thereof. In some embodiments, the pharmaceutically effective amount of the FKBP ligand is about 1 mg to about 1,000 mg, for example about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, or about 1,000 mg.

In some embodiments, the composition includes more than one FKBP ligand or derivative thereof. In such embodiments, the total amount of FKBP ligands and derivatives thereof may be about 1 mg to about 1,000 mg, for example about 11 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, or about 1,000 mg. In some embodiments, the composition (e.g., pharmaceutical composition) further includes one or more antiviral agents. In some embodiments, the antiviral agent is selected from the group consisting of: remdesivir, molnupiravir, favipiravir (Avigan), AT-527, triazavirin (riamilovir), ribavirin, plitidepsin, danoprevir (Prezcobix), ASC09F (also referred to as TMC-310911), tenofovir, atazanavir, oseltamivir (Tamiflu), emtricitabine, PF-07304814, PF-07321332, S-217622, and daclatasvir. In some embodiments, the antiviral agent is an RNA polymerase inhibitor, such as remdesivir. In some embodiments, the antiviral agent is molnupiravir (also referred to as MK-4482, EIDD-2801, or ((2R,3S,4R,5R)-3,4-dihydroxy-5-(4-(hydroxyimino)-2-oxo-3,4-dihydropyrimidin-1(2H)-yl)tetrahydrofuran-2-yl)methyl isobutyrate). In some embodiments, the one or more antiviral agents are present in an amount less than the known minimum therapeutic amount necessary if the antiviral agent(s) were to be administered without an FKBP ligand or derivative thereof.

In some embodiments, the composition (e.g., pharmaceutical composition) further includes one or more protease inhibitors. In some embodiments, the protease inhibitor is M128533. In some embodiments, the protease inhibitor is a 3CL protease inhibitor such as S-217622. In some embodiments, the one or more protease inhibitors are present in an amount less than the known minimum therapeutic amount necessary if the protease inhibitor(s) were to be administered without an FKBP ligand or derivative thereof.

Various embodiments can, if desired, include one or more pharmaceutically acceptable excipients. The term “excipient” herein means any substance, not itself a therapeutic agent, used as a carrier or vehicle for delivery of an FKBP ligand or derivative thereof to a subject or added to a composition to improve its handling or storage properties or to permit or facilitate formation of a dose unit of the composition. Excipients include, by way of illustration and not limitation, diluents, disintegrants, binding agents, adhesives, wetting agents, lubricants, glidants, surface modifying agents, substances added to mask or counteract a disagreeable taste or odor, flavors, dyes, fragrances, and substances added to improve appearance of the composition. Any such excipients can be used in any dosage forms according to the present disclosure, including liquid, solid or semi-solid dosage forms.

Excipients optionally employed in various embodiments can be solids, semi-solids, liquids or combinations thereof. Compositions of the disclosure including excipients can be prepared by various pharmaceutical techniques such as admixing an excipient with a drug or therapeutic agent.

In various embodiments, compositions consistent with the present disclosure may optionally comprise one or more pharmaceutically acceptable diluents as excipients. Suitable diluents illustratively include, without limitation, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of a- and amorphous cellulose (e.g., Rexcel™) and powdered cellulose; calcium carbonate; glycine; bentonite; polyvinylpyrrolidone; and the like. Such diluents, if present, may constitute in total about 5% to about 99%, about 10% to about 85%, or about 20% to about 80%, of the total weight of the composition. In various embodiments, the diluent or diluents selected may exhibit suitable flow properties and, where tablets are desired, compressibility.

The use of extragranular microcrystalline cellulose (that is, microcrystalline cellulose added to a wet granulated composition after a drying step) can be used to alter or control hardness (for tablets) and/or disintegration time.

In various embodiments, compositions consistent with the present disclosure may optionally comprise one or more pharmaceutically acceptable disintegrants as excipients, such as in tablet formulations. Suitable disintegrants include, without limitation, either individually or in combination, starches, including crosslinked polyvinylpyrrolidone (crospovidone USP/NF), carboxymethyl cellulose (sodium CMC), chitin, chitosan, sodium starch glycolate (e.g., Explotab™ of PenWest) and pregelatinized corn starches (e.g., National™ 1551, National™ 1550, and Colocorn™ 1500), clays (e.g., Veegum™ HV), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, and gums such as agar, guar, xanthan, locust bean, karaya, pectin and tragacanth gums.

Disintegrants may be added at any suitable step during the preparation of the composition, particularly prior to a granulation step or during a lubrication step prior to compression. Such disintegrants, if present, may constitute in total about 0.2% to about 30%, about 0.2% to about 10%, or about 0.2% to about 5%, of the total weight of the composition.

In one embodiment, crosslinked polyvinylpyrrolidone (crospovidone USP/NF) is an optional disintegrant for tablet or capsule disintegration, and, if present, may optionally constitute about 1% to about 5% of the total weight of the composition.

In another embodiment, chitin is an optional disintegrant for tablet or capsule disintegration.

In still another embodiment, chitosan is an optional disintegrant for tablet or capsule disintegration.

In still another embodiment, carboxymethyl cellulose (sodium CMC) is an optional disintegrant for tablet or capsule disintegration.

In another embodiment, croscarmellose sodium is a disintegrant for tablet or capsule disintegration, and, if present, may optionally constitute about 0.2% to about 10%, about 0.2% to about 7%, or about 0.2% to about 5%, of the total weight of the composition.

Various embodiments consistent with the present disclosure may optionally comprise one or more pharmaceutically acceptable binding agents or adhesives as excipients, particularly for tablet formulations. Such binding agents and adhesives may impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, without limitation, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose (e.g., Klucel™); and ethylcellulose (e.g., Ethocel™). Such binding agents and/or adhesives, if present, may constitute in total about 0.5% to about 25%, about 0.75% to about 15%, or about 1% to about 10%, of the total weight of the composition.

Compositions consistent with the present disclosure may optionally comprise one or more pharmaceutically acceptable wetting agents as excipients. Non-limiting examples of surfactants that can be used as wetting agents in various compositions include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefossé), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefossé), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, may constitute in total about 0.25% to about 15%, about 0.4% to about 10%, or about 0.5% to about 5%, of the total weight of the composition.

Compositions consistent with the present disclosure may optionally comprise one or more pharmaceutically acceptable lubricants (including anti-adherents and/or glidants) as excipients. Suitable lubricants include, without limitation, either individually or in combination, glyceryl behapate (e.g., Compritol™ 888); stearic acid and salts thereof, including magnesium (magnesium stearate), calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; PEG (e.g., Carbowax™ 4000 and Carbowax™ 6000); sodium oleate; sodium lauryl sulfate; and magnesium lauryl sulfate. Such lubricants, if present, may constitute in total about 0.1% to about 10%, about 0.2% to about 8%, or about 0.25% to about 5%, of the total weight of the composition.

Suitable anti-adherents include, without limitation, talc, cornstarch, DL-leucine, sodium lauryl sulfate and metallic stearates. Talc is a anti-adherent or glidant used, for example, to reduce formulation sticking to equipment surfaces and also to reduce static in the blend. Talc, if present, may constitute about 0.1% to about 10%, about 0.25% to about 5%, or about 0.5% to about 2%, of the total weight of the composition.

Glidants can be used to promote powder flow of a solid formulation. Suitable glidants include, without limitation, colloidal silicon dioxide, starch, talc, tribasic calcium phosphate, powdered cellulose and magnesium trisilicate.

Compositions consistent with the present disclosure may can comprise one or more flavoring agents, sweetening agents, and/or colorants. Flavoring agents useful in the present embodiments include, without limitation, acacia syrup, alitame, anise, apple, aspartame, banana, Bavarian cream, berry, black currant, butter, butter pecan, butterscotch, calcium citrate, camphor, caramel, cherry, cherry cream, chocolate, cinnamon, citrus, citrus punch, citrus cream, cocoa, coffee, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, MagnaSweet®, maltol, mannitol, maple, menthol, mint, mint cream, mixed berry, nut, orange, peanut butter, pear, peppermint, peppermint cream, Prosweet® Powder, raspberry, root beer, rum, saccharin, safrole, sorbitol, spearmint, spearmint cream, strawberry, strawberry cream, stevia, sucralose, sucrose, Swiss cream, tagatose, tangerine, thaumatin, tutti fruitti, vanilla, walnut, watermelon, wild cherry, wintergreen, xylitol, and combinations thereof, for example, anise-menthol, cherry-anise, cinnamon-orange, cherry-cinnamon, chocolate-mint, honey-lemon, lemon-lime, lemon-mint, menthol-eucalyptus, orange-cream, vanilla-mint, etc.

Sweetening agents that can be used in the present embodiments include, by way of example and not limitation, acesulfame potassium (acesulfame K), alitame, aspartame, cyclamate, cylamate, dextrose, isomalt, MagnaSweet®, maltitol, mannitol, neohesperidine DC, neotame, Prosweet0 Powder, saccharin, sorbitol, stevia, sucralose, sucrose, tagatose, thaumatin, xylitol, and the like.

The foregoing excipients can have multiple roles. For example, starch can serve as a filler as well as a disintegrant. The classification of excipients listed herein is not to be construed as limiting in any manner.

Compositions consistent with the present disclosure may comprise one or more pharmaceutically acceptable buffering agents. Buffering agents useful in accordance with the present disclosure may be one or more agents possessing pharmacological activity as a weak or strong base. In some embodiments, a buffering agent may be a salt of a Group IA metal including, for example, a bicarbonate salt of a Group IA metal, a carbonate salt of a Group IA metal, an alkaline earth metal buffering agent, an amino acid, an alkaline salt of an ammo acid, an aluminum buffering agent, a calcium buffering agent, a sodium buffering agent, or a magnesium buffering agent. Other suitable buffering agents include alkali (sodium and potassium) or alkaline earth (calcium and magnesium) carbonates, phosphates, bicarbonates, citrates, borates, acetates, phthalates, tartrates, succinates and the like, such as sodium or potassium phosphate, citrate, borate, acetate, bicarbonate and carbonate.

Non-limiting examples of suitable buffering agents include aluminum, magnesium hydroxide, aluminum hydroxide/magnesium hydroxide co-precipitate, aluminum hydroxide/sodium bicarbonate co-precipitate, calcium acetate, calcium bicarbonate, calcium borate, calcium carbonate, calcium bicarbonate, calcium citrate, calcium gluconate, calcium glycerophosphate, calcium hydroxide, calcium lactate, calcium phthalate, calcium phosphates (including Calcium dihydrogen phosphate, tricalcium phosphate and the like), calcium succinate, calcium tartrate, calcium formate, calcium propionate, dibasic sodium phosphate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, and trometamol. (Based in part upon the list provided in The Merck Index, Merck & Co. Rahway, N.J. (2001)). In addition, due to the ability of proteins or protein hydrolysates to react with stomach acids, they too can serve as buffering agents in the present embodiments. Furthermore, combinations or mixtures of the above-mentioned buffering agents can be used in the compositions described herein.

Compositions of the present disclosure can be in the form of an orally deliverable dosage unit. The terms “oral administration” or “orally deliverable” herein include any form of delivery of a therapeutic agent or a composition thereof to a subject wherein the agent or composition is placed in the mouth of the subject, whether or not the agent or composition is swallowed. Thus, “oral administration” includes buccal and sublingual as well as esophageal administration. Such dosage units may be administered one to a plurality (i.e. 1 to about 10, 1 to 8, 1 to 6, 1 to 4 or 1 to 2) of times per day, or as many times as needed to elicit a therapeutic response.

Compositions of the present disclosure may be in the form of a parenterally dosage form, such as a solution or a mixture. Such dosage units may be administered one to a plurality (i.e. 1 to about 10, 1 to 8, 1 to 6, 1 to 4 or 1 to 2) of times per day or per week, or as many times as needed to elicit a therapeutic response. In some embodiments, the parenteral dosage form is in an injectable form. In some embodiments, the parental dosage form is in a form suitable for slow administration via an intravenous line.

Compositions of the present disclosure may be in an aerosolizeable form, such as a powder or a mist, or a liquid form configured to be administered by nebulizer. Such dosage forms may include a carrier, such as a solid carrier or a liquid carrier. The solid carrier (e.g., for a powdered dosage form) may be any suitable solid carrier, for example and without limitation lactose monohydrate (e.g., lactose monohydrate 200 mesh and/or micronized lactose), and/or anhydrous glucose (e.g., anhydrous glucose 35 μm and/or micronized anhydrous glucose). Powder dosage forms may further comprise one or more encapsulation excipients, such as one or more monosaccharide (e.g., glucose and/or arabinose), one or more disaccharide (e.g., lactose, trehalose, sucrose, and/or maltose), one or more oligo- and/or polysaccharide (e.g., dextranes and/or hydroxyethyl cellulose), one or more polyalcohol (e.g., sorbitol, mannitol, and/or xylitol), one or more salt (e.g., sodium chloride and/or calcium carbonate), one or more polyester (e.g., polylactides and/or one or more polylactide copolymer), one or more polyether (e.g., one or more PEG species), and/or one or more sugar ester, ether, and/or polyvinyl derivatives (e.g., one or more polyvinyl alcohol species). A powder dosage form may have an average geometric particle size of about 0.5 μm to about 25 μm, for example about 0.5 μm, about 1 μm, about 1.5 μm, about 2 μm, about 2.5 μm, about 3 μm, about 3.5 μm, about 4 μm, about 4.5 μm, about 5 μm, about 5.5 μm, about 6 μm, about 6.5 μm, about 7 μm, about 7.5 μm, about 8 μm, about 8.5 μm, about 9 μm, about 9.5 μm, about 10 μm, about 10.5 μm, about 11 μm, about 11.5 μm, about 12 μm, about 12.5 μm, about 13 μm, about 13.5 μm, about 14 μm, about 14.5 μm, about 15 μm, about 15.5 μm, about 16 μm, about 16.5 μm, about 17 μm, about 17.5 μm, about 18 μm, about 18.5 μm, about 19 μm, about 19.5 μm, about 20 μm, about 20.5 μm, about 21 μm, about 21.5 μm, about 22 μm, about 22.5 μm, about 23 μm, about 23.5 μm, about 24 μm, about 24.5 μm, or about 25 μm.

In some embodiments, a composition for parenteral administration (e.g., by IV or injection) includes an FKBP ligand or derivative thereof and a parenteral carrier (e.g., a solvent). In some embodiments, the solvent comprises, consists essentially of, or consists of normal saline or 5% dextrose in water. In some embodiments, the parenteral carrier further includes a solubilizer, such as polyoxyl 60 hydrogenated castor oil (HCO-60). In other embodiments, the parenteral composition includes the FKBP ligand in nanoparticulate form (e.g., having an effective average particle size of less than about 600 nm and at least one surface stabilizer that is a povidone polymer) and does not include HCO-60.

In some embodiments, a composition for parenteral administration consistent with the present disclosure includes the FKBP ligand or derivative thereof at a concentration of about 1 mg/ml to about 10 mg/mL, for example about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, or about 10 mg/mL. In some embodiments, the composition for parenteral administration consistent with the present disclosure includes the FKBP ligand or derivative thereof at a concentration of about 0.004 mg/mL to about 0.02 mg/mL, for example 0.004 mg/mL, about 0.005 mg/mL, about 0.006 mg/mL, about 0.007 mg/mL, about 0.008 mg/mL, about 0.009 mg/mL, about 0.01 mg/mL, about 0.011 mg/mL, about 0.012 mg/mL, about 0.013 mg/mL, about 0.014 mg/mL, about 0.015 mg/mL, about 0.016 mg/mL, about 0.017 mg/mL, about 0.018 mg/mL, about 0.019 mg/mL, or about 0.02 mg/mL.

In some embodiments, a composition for aerosol administration consistent with the present disclosure includes an FKBP ligand or derivative thereof and a propellant. The propellant may include liquids such as liquid chlorofluorocarbons (CFCs) or liquid fluorocarbons (also referred to as hydrofluoroalkanes or HFAs).

Nonlimiting examples of CFC propellants include CCl₃F (CFC-11), CCl₂F₂ (CFC-12), C₂Cl₂F₄ (CFC-114), and mixtures thereof. Nonlimiting examples of suitable HFAs (also referred to as HFCs) include 1,1,1,2-tetrafluoroethane (HFC-134a) and 1,1,1,2,3,3,3-heptafluoropropane (HFC-227ea, also referred to as HFC-227), difluoromethane (HFC-32), 1,1,1-trifluoroethane (HFC-143(a)), 1,1,2,2-tetrafluoroethane (HFC-134), 1,1-difluoroethane (HFC-152a), and mixtures thereof. In some embodiments, the propellant includes dimethyl ether (DME).

In some embodiments, an aerosolizable composition consistent with the present disclosure comprise a co-solvent. The term “co-solvent” as used herein refers to a substance, such as a liquid, in which other substances are dissolved. Co-solvents, as employed herein, are typically less volatile than propellants and are used to help dissolve a drug such as an FKBP ligand or derivative thereof or other excipients in a propellant, lower the vapor pressure of the propellant system, and/or promote miscibility between propellants and immiscible solvents. Generally, a co-solvent as provided herein is an organic molecule that possesses a molecular weight of less than about 150, and a boiling point of less than about 200° C. In some embodiments, the co-solvent has a boiling point of less than about 100° C. Non-limiting examples of co-solvents suitable for use in an aerosolizeable composition consistent with the present disclosure include, but are not limited to, alcohols (e.g., ethanol, isopropyl alcohol, propanol, tert-butyl alcohol, propylene glycol, and the like). In some embodiments, the co-solvent is ethanol.

The co-solvent may be present in an amount of about 1-10 wt. % of the total composition mass, for example about 1 wt. %, about 2 wt. %, about 3 wt. %, about 4 wt. %, about 5 wt. %, about 6 wt. %, about 7 wt. %, about 8 wt. %, about 9 wt. %, or about 10 wt. % on a weight percentage basis of the formulation.

Aerosolizeable compositions provided herein are suitable for aerosol administration and in some embodiments comprise an FKBP ligand or derivative thereof dissolved in a co-solvent-liquified propellant mixture, for example an ethanol-liquified propellant mixture. In some embodiments, a concentration of the FKBP ligand or derivative thereof is greater than about 0.15 wt. % (% w/w). In some embodiments, the co-solvent is present at a concentration of not more than about 10 wt. % (% w/w).

Additionally, the solutions provided herein will typically possess a volume ratio of co-solvent to FKBP ligand or derivative thereof of about 50 to about 500 mg/mL, for example about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, about 210 mg/mL, about 220 mg/mL, about 230 mg/mL, about 240 mg/mL, about 250 mg/mL, about 260 mg/mL, about 270 mg/mL, about 280 mg/mL, about 290 mg/mL, about 300 mg/mL, about 310 mg/mL, about 320 mg/mL, about 330 mg/mL, about 340 mg/mL, about 350 mg/mL, about 360 mg/mL, about 370 mg/mL, about 380 mg/mL, about 390 mg/mL, about 400 mg/mL, about 410 mg/mL, about 420 mg/mL, about 430 mg/mL, about 440 mg/mL, about 450 mg/mL, about 460 mg/mL, about 470 mg/mL, about 480 mg/mL, about 490 mg/mL, or about 500 mg/mL.

Aerosolizeable compositions consistent with the present disclosure may further include a solubility enhancer, for example a solubility enhancer soluble in the propellant. Nonlimiting examples of solubility enhancers suitable for inclusion in aerosolizeable compositions of the present disclosure include oligolactic acids, acyl amide acids, monofunctionalized methoxypolyethylene glycols (mPEGs) and acylated α, β, or γ-cyclodextrins.

In some embodiments, the aerosolizeable composition does not include a triglyceride.

Compositions (e.g., pharmaceutical compositions) consistent with the present disclosure do not suppress an immune response in the subject upon administration. The term “non-immunosuppressive” may be used to describe such compositions. The phrase “do not suppress an immune response” as used herein means that, upon administration of the composition, the subject's immune system is not substantially suppressed, is not significantly suppressed (e.g., compared to other subjects receiving the composition), or is not totally suppressed.

In some embodiments, the present disclosure provides a pharmaceutical composition comprising an FKBP ligand or derivative thereof. In some embodiments, the FKBP ligand or derivative thereof is one or more of: tacrolimus, everolimus, sirolimus, 15-O-demethyl tacrolimus, GPI1046, GPI-1485, ElteN378, V10367, L-685818, WAY 124466, AG5473, AG5507, meridamycin, and FK1706. In some embodiments, the pharmaceutical composition further comprises an antiviral agent. In some embodiments, the antiviral agent is selected from the group consisting of: remdesivir, molnupiravir, favipiravir (Avigan), AT-527, triazavirin (riamilovir), ribavirin, plitidepsin, danoprevir (Prezcobix), ASC09F (also referred to as TMC-310911), tenofovir, atazanavir, oseltamivir (Tamiflu), emtricitabine, PF-07304814, PF-07321332, S-217622, and daclatasvir. In some embodiments, the antiviral agent is an RNA polymerase inhibitor. In some embodiments, the RNA polymerase inhibitor is remdesivir. In some embodiments, the RNA polymerase inhibitor is molnupiravir. In some embodiments, the pharmaceutical composition further comprises a protease inhibitor. In some embodiments, the protease inhibitor is M128533. In some embodiments, the pharmaceutical composition does not substantially suppress an immune response in a subject who has been administered the pharmaceutical composition.

In some embodiments, the present disclosure provides a pharmaceutical composition comprising 15-O-demethyl tacrolimus, wherein the pharmaceutical composition does not substantially suppress an immune response in a subject who has been administered the pharmaceutical composition.

In some embodiments, the present disclosure provides a pharmaceutical composition comprising an FKBP ligand or a derivative thereof; and an antiviral agent. In some embodiments, the FKBP ligand or derivative thereof is one or more of: tacrolimus, everolimus, sirolimus, 15-O-demethyl tacrolimus, GPI1046, GPI-1485, ElteN378, V10367, L-685818, WAY 124466, AG5473, AG5507, meridamycin, and FK1706. In some embodiments, the antiviral agent is an RNA polymerase inhibitor. In some embodiments, the antiviral agent is present in a sub-therapeutic amount. In some embodiments, the RNA polymerase inhibitor is remdesivir. In some embodiments, the remdesivir is present in an amount not more than a loading dose (e.g., not more than about 200 mg for a subject aged 12 or older). In some embodiments, the remdesivir is present in an amount not more than a maintenance dose (e.g., not more than about 100 mg for a subject aged 12 or older). In some embodiments, the RNA polymerase inhibitor is molnupiravir. In some embodiments, the antiviral agent is a protease inhibitor. In some embodiments, the protease inhibitor is M128533. In some embodiments, the FKBP ligand or derivative thereof is present in a sub-therapeutic amount. In some embodiments, the FKBP ligand or derivative thereof is FK1706.

In some embodiments, the present disclosure provides a pharmaceutical composition comprising: a sub-therapeutic amount of an FKBP ligand or a derivative thereof; and a sub-therapeutic amount of an antiviral agent. In some embodiments, the FKBP ligand or derivative thereof is one or more of: tacrolimus, everolimus, sirolimus, 15-O-demethyl tacrolimus, GPI1046, GPI-1485, ElteN378, V10367, L-685818, WAY 124466, AG5473, AG5507, meridamycin, and FK1706. In some embodiments, the antiviral agent is selected from the group consisting of: remdesivir, molnupiravir, favipiravir (Avigan), AT-527, triazavirin (riamilovir), ribavirin, plitidepsin, danoprevir (Prezcobix), ASC09F (also referred to as TMC-310911), tenofovir, atazanavir, oseltamivir (Tamiflu), emtricitabine, PF-07304814, PF-07321332, S-217622, and daclatasvir. In some embodiments, the antiviral agent is an RNA polymerase inhibitor. In some embodiments, the RNA polymerase inhibitor is remdesivir. In some embodiments, the sub-therapeutic amount of remdesivir is not more than a loading dose (e.g., about 200 mg for a subject aged 12 or older). In some embodiments, the sub-therapeutic amount of remdesivir is not more than a maintenance dose (e.g., about 100 mg for a subject aged 12 or older). In some embodiments, the RNA polymerase inhibitor is molnupiravir. In some embodiments, the molnupiravir is co-administered to the subject at a sub-therapeutic dose. In some embodiments, the sub-therapeutic dose of molnupiravir is not more than a loading dose. In some embodiments, the sub-therapeutic dose of molnupiravir is not more than a maintenance dose In some embodiments, the antiviral agent is a protease inhibitor. In some embodiments, the protease inhibitor is M128533. In some embodiments, the FKBP ligand or derivative thereof is FK1706.

### Methods of Treating Coronavirus Infections

The present disclosure provides methods of treating coronavirus infections in a subject (e.g., a human subject or patient).

In some embodiments, the present disclosure provides methods of treating a coronavirus infection in a subject in need thereof, the method comprising identifying a coronavirus infection in the subject and, thereafter, administering to the subject a composition comprising an FKBP ligand or derivative thereof. In some embodiments, the step of administering the composition comprises administering to the subject a therapeutically effective amount of the composition.

The step of identifying the coronavirus infection in the subject may be performed by any suitable means, such as a blood test and/or an antigen test.

The step of administering the composition to the subject does not suppress an immune response in the subject. In some embodiments, the immune response of the subject is not substantially suppressed. In some embodiments, the immune response of the subject is not significantly suppressed (e.g., compared to other subjects receiving the composition). In some embodiments, the immune response of the subject is not totally suppressed.

In one embodiment, the method comprises administering a pharmaceutical composition as disclosed herein once per day, twice per day, three times per day, or more than three times per day.

In some embodiments, the step of administering the composition comprises orally administering the composition to the subject. In some embodiments, the composition is orally administered one to a plurality (i.e. 1 to about 10, 1 to 8, 1 to 6, 1 to 4 or 1 to 2) of times per day, or as many times as needed to elicit a therapeutic response.

In some embodiments, the step of administering the composition comprises parenterally administering the composition to the subject, for example by IV or injection. In some embodiments, the composition is parenterally administered one to a plurality (i.e. 1 to about 10, 1 to 8, 1 to 6, 1 to 4 or 1 to 2) of times per day or per week, or as many times as needed to elicit a therapeutic response. In some embodiments, the composition is parenterally administered by injection. In some embodiments, the composition is parenterally administered by intravenous line. In some embodiments, the FKBP ligand or derivative thereof is administered parenterally at a concentration of about 0.004 mg/mL to about 0.02 mg/mL.

In some embodiments, the step of administering the composition comprises administering the composition to the subject by aerosol, for example by metered dose spray or by nebulizer. In some embodiments, the composition is administered by aerosol one to a plurality (i.e. 1 to about 10, 1 to 8, 1 to 6, 1 to 4 or 1 to 2) of times per day or per week, or as many times as needed to elicit a therapeutic response.

In some embodiments, the present disclosure provides a method of treating a coronavirus infection in a subject in need thereof, the method comprising: identifying a coronavirus infection in a subject; and thereafter administering a pharmaceutical composition comprising an FKBP ligand or derivative thereof to the subject. In some embodiments, the FKBP ligand or derivative thereof is one or more of: tacrolimus, everolimus, sirolimus, 15-O-demethyl tacrolimus, GPI1046, GPI-1485, ElteN378, V10367, L-685818, WAY 124466, AG5473, AG5507, meridamycin, and FK1706. In some embodiments, the pharmaceutical composition further comprises an antiviral agent. In some embodiments, the antiviral agent is selected from the group consisting of: remdesivir, molnupiravir, favipiravir (Avigan), AT-527, triazavirin (riamilovir), ribavirin, plitidepsin, danoprevir (Prezcobix), ASC09F (also referred to as TMC-310911), tenofovir, atazanavir, oseltamivir (Tamiflu), emtricitabine, PF-07304814, PF-07321332, S-217622, and daclatasvir. In some embodiments, the antiviral agent is an RNA polymerase inhibitor. In some embodiments, the RNA polymerase inhibitor is remdesivir. In some embodiments, the RNA polymerase inhibitor is molnupiravir. In some embodiments, the pharmaceutical composition further comprises a protease inhibitor. In some embodiments, the protease inhibitor is M128533. In some embodiments, an immune response associated with the subject is not substantially suppressed after the step of administering the pharmaceutical composition to the subject.

In some embodiments, the present disclosure provides a method of treating a coronavirus infection in a subject in need thereof, the method comprising: identifying a coronavirus infection associated with the subject; and thereafter administering to the subject a pharmaceutical composition comprising 15-O-demethyl tacrolimus, wherein an immune response associated with the subject is not substantially suppressed after the step of administering the pharmaceutical composition to the subject.

In some embodiments, the present disclosure provides a method of treating or preventing a coronavirus infection in a subject in need thereof, the method comprising co-administering to the subject in need thereof an FKBP ligand or derivative thereof and an antiviral agent. In some embodiments, the FKBP ligand or derivative thereof is one or more of: tacrolimus, everolimus, sirolimus, 15-O-demethyl tacrolimus, GPI1046, GPI-1485, ElteN378, V10367, L-685818, WAY 124466, AG5473, AG5507, meridamycin, and FK1706. In some embodiments, the antiviral agent is selected from the group consisting of: remdesivir, molnupiravir, favipiravir (Avigan), AT-527, triazavirin (riamilovir), ribavirin, plitidepsin, danoprevir (Prezcobix), ASC09F (also referred to as TMC-310911), tenofovir, atazanavir, oseltamivir (Tamiflu), emtricitabine, PF-07304814, PF-07321332, S-217622, and daclatasvir. In some embodiments, the antiviral agent is an RNA polymerase inhibitor. In some embodiments, the RNA polymerase inhibitor is remdesivir. In some embodiments, the remdesivir is co-administered to the subject at a sub-therapeutic dose. In some embodiments, the sub-therapeutic dose of remdesivir is not more than a loading dose (e.g., not more than about 200 mg to a subject aged 12 or older). In some embodiments, the sub-therapeutic dose of remdesivir is not more than a maintenance dose (e.g., not more than about 100 mg to a subject aged 12 or older). In some embodiments, the RNA polymerase inhibitor is molnupiravir. In some embodiments, the molnupiravir is co-administered to the subject at a sub-therapeutic dose. In some embodiments, the sub-therapeutic dose of molnupiravir is not more than a loading dose. In some embodiments, the sub-therapeutic dose of molnupiravir is not more than a maintenance dose. In some embodiments, the antiviral agent is a protease inhibitor. In some embodiments, the protease inhibitor is M128533. In some embodiments, the FKBP ligand or derivative thereof is FK1706. In some embodiments, the method is a method of preventing a coronavirus in a subject who does not have a diagnosed coronavirus infection. In some embodiments, the method is a method of treating an existing coronavirus infection in the subject. In some embodiments, the method further comprises identifying a coronavirus infection in the subject in need thereof before the step of co-administering the FKBP ligand or derivative thereof and the antiviral agent to the subject. In some embodiments, an immune response associated with the subject is not substantially suppressed after the step of co-administering the FKBP ligand or derivative thereof and the antiviral agent to the subject. In some embodiments, the coronavirus infection is a SARS COV-2 viral infection. In some embodiments, the FKBP ligand is administered to the subject at a sub-therapeutic dose.

In some embodiments, the present disclosure provides a method of treating or preventing a coronavirus infection in a subject in need thereof, the method comprising co-administering to the subject in need thereof a sub-therapeutic amount of an FKBP ligand or derivative thereof and a sub-therapeutic amount of an antiviral agent. In some embodiments, the FKBP ligand or derivative thereof is one or more of: tacrolimus, everolimus, sirolimus, 15-O-demethyl tacrolimus, GPI1046, GPI-1485, ElteN378, V10367, L-685818, WAY 124466, AG5473, AG5507, meridamycin, and FK1706. In some embodiments, the antiviral agent is selected from the group consisting of: remdesivir, molnupiravir, favipiravir (Avigan), AT-527, triazavirin (riamilovir), ribavirin, plitidepsin, danoprevir (Prezcobix), ASC09F (also referred to as TMC-310911), tenofovir, atazanavir, oseltamivir (Tamiflu), emtricitabine, PF-07304814, PF-07321332, S-217622, and daclatasvir. In some embodiments, the antiviral agent is an RNA polymerase inhibitor. In some embodiments, the RNA polymerase inhibitor is remdesivir. In some embodiments, the sub-therapeutic amount of remdesivir is not more than a loading dose (e.g., about 200 mg for a subject aged 12 or older). In some embodiments, the sub-therapeutic amount of remdesivir is not more than a maintenance dose (e.g., about 100 mg for a subject aged 12 or older). In some embodiments, the RNA polymerase inhibitor is molnupiravir. In some embodiments, the sub-therapeutic amount of molnupiravir is not more than a loading dose. In some embodiments, the sub-therapeutic amount of molnupiravir is not more than a maintenance dose. In some embodiments, the antiviral agent is a protease inhibitor. In some embodiments, the protease inhibitor is M128533. In some embodiments, the FKBP ligand or derivative thereof is FK1706. In some embodiments, the method is a method of preventing a coronavirus in a subject who does not have a diagnosed coronavirus infection. In some embodiments, the method is a method of treating an existing coronavirus infection in the subject. In some embodiments, the method of treating an existing coronavirus infection further comprises identifying a coronavirus infection in the subject in need thereof before the step of co-administering the FKBP ligand and the antiviral agent to the subject. In some embodiments, an immune response associated with the subject is not substantially suppressed after the step of co-administering the FKBP ligand and the antiviral agent to the subject. In some embodiments, the coronavirus infection is a SARS COV-2 viral infection.

In some embodiments, the present disclosure provides a method of treating or preventing a coronavirus infection in a subject in need thereof, the method comprising co-administering to the subject in need thereof an FKBP ligand or derivative thereof and an antiviral agent. In some embodiments, the FKBP ligand or derivative thereof is one or more of: tacrolimus, everolimus, sirolimus, 15-O-demethyl tacrolimus, GPI1046, GPI-1485, ElteN378, V10367, L-685818, WAY 124466, AG5473, AG5507, meridamycin, and FK1706. In some embodiments, the antiviral agent is selected from the group consisting of: favipiravir (Avigan), AT-527, triazavirin (riamilovir), ribavirin, plitidepsin, danoprevir (Prezcobix), ASC09F (also referred to as TMC-310911), tenofovir, atazanavir, oseltamivir (Tamiflu), emtricitabine, PF-07304814, PF-07321332, S-217622, and daclatasvir. In some embodiments, the antiviral agent is an RNA polymerase inhibitor. In some embodiments, the RNA polymerase inhibitor is remdesivir. In some embodiments, the RNA polymerase inhibitor is molnupiravir. In some embodiments, the sub-therapeutic amount of the antiviral agent is a sub-therapeutic amount. For example, in embodiments wherein the antiviral agent is remdesivir, the remdesivir may be co-administered with the FKBP ligand in an amount not exceeding a loading dose (e.g., not more than about 200 mg or remdesivir for a subject aged 12 or older), and/or in an amount not exceeding a maintenance dose (e.g., not more than about 100 mg of remdesivir for a subject aged 12 or older). In embodiments wherein the antiviral agent is molnupiravir, the molnupiravir may be co-administered with the FKBP ligand in an amount not exceeding a loading dose, and/or in an amount not exceeding a maintenance dose. In some embodiments, the antiviral agent is a protease inhibitor. In some embodiments, the protease inhibitor is M128533. In some embodiments, the FKBP ligand or derivative thereof is FK1706. In some embodiments, the method is a method of preventing a coronavirus in a subject who does not have a diagnosed coronavirus infection. In some embodiments, the method is a method of treating an existing coronavirus infection in the subject. In some embodiments, the method of treating an existing coronavirus infection further comprises identifying a coronavirus infection in the subject in need thereof before the step of co-administering the FKBP ligand and the antiviral agent to the subject. In some embodiments, an immune response associated with the subject is not substantially suppressed after the step of co-administering the FKBP ligand and the antiviral agent to the subject. In some embodiments, the coronavirus infection is a SARS COV-2 viral infection.

In some embodiments, the co-administration of the FKBP ligand and the antiviral agent results in improved clinical outcome(s) in a subject compared to clinical outcome(s) for a control subject receiving the FKBP ligand as a monotherapy (e.g., at a similar dose to the amount of FKBP ligand co-administered to the subject). For example and without limitation, the improved clinical outcome(s) may include one or more of: shortened duration of coronavirus-related symptoms, reduced severity of coronavirus-related symptoms, reduced risk of hospitalization, reduced duration of hospitalization, reduced risk of death from coronavirus, reduced risk of permanent tissue or organ damage due to coronavirus, reduced risk of respiratory distress due to coronavirus, reduced risk of requirement for ventilator intervention due to coronavirus, reduced risk of loss of sense of smell due to coronavirus, reduced risk of loss of taste due to coronavirus, reduced incidence of a side effect associated with administration of the FKBP ligand as a monotherapy, and/or reduced severity of a side effect associated with administration of the FKBP ligand as a monotherapy.

In some embodiments, the co-administration of the FKBP ligand and the antiviral agent results in improved clinical outcome(s) in a subject compared to clinical outcome(s) for a control subject receiving the antiviral agent as a monotherapy (e.g., at a similar dose to the amount of antiviral agent co-administered to the subject). For example and without limitation, the improved clinical outcome(s) may include one or more of: shortened duration of coronavirus-related symptoms, reduced severity of coronavirus-related symptoms, reduced risk of hospitalization, reduced duration of hospitalization, reduced risk of death from coronavirus, reduced risk of permanent tissue or organ damage due to coronavirus, reduced risk of respiratory distress due to coronavirus, reduced risk of requirement for ventilator intervention due to coronavirus, reduced risk of loss of sense of smell due to coronavirus, reduced risk of loss of taste due to coronavirus, reduced incidence of a side effect associated with administration of the antiviral agent as a monotherapy, and/or reduced severity of a side effect associated with administration of the antiviral agent as a monotherapy.

In some embodiments, methods of the present disclosure result in reduced incidence of secondary infections (e.g., viral infections other than coronavirus) in subjects administered a composition as disclosed herein, compared to secondary infection incident rates associated with similar subjects that receive compositions including only tacrolimus, only everolimus, or only sirolimus.

In some embodiments, a method of treatment consistent with the present disclosure results in subjects in a treated subject group exhibiting a reduced hospitalization rate from a coronavirus infection or a coronavirus-related symptom, relative to subjects in a comparative subject group that are not treated by the same method (e.g., is treated by an antiviral monotherapy, by a FKBP ligand monotherapy, by a placebo, or receives no therapy). In some embodiments, the reduced hospitalization rate is at least about 5% relative to the comparative subject group, for example at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater than 99% relative to the comparative subject group. In some embodiments, the hospitalization event comprises acute care in a hospital or acute care facility for at least about 24 hours.

In some embodiments, a method of treatment consistent with the present disclosure results in subjects in a treated subject group exhibiting a reduced death rate after a coronavirus infection relative to subjects in a comparative subject group that are not treated by the same method (e.g., is treated by an antiviral monotherapy, by a FKBP ligand monotherapy, by a placebo, or receives no therapy). In some embodiments, the reduced death rate after a coronavirus infection is at least about 5% relative to the comparative subject group, for example at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater than 99% relative to the comparative subject group.

In some embodiments, a method of treatment consistent with the present disclosure results in a smaller (e.g., substantially smaller) portion (e.g., fraction or percentage) of subjects in a treated subject group experiencing an adverse event relative to subjects in a comparative subject group that are not treated by the same method (e.g., is treated by an antiviral monotherapy, by a FKBP ligand monotherapy, by a placebo, or receives no therapy). In some embodiments, the smaller portion is not more than about 95% relative to the comparative subject group, for example not more than about 95%, not more than about 94%, not more than about 93%, not more than about 92%, not more than about 91%, not more than about 90%, not more than about 89%, not more than about 88%, not more than about 87%, not more than about 86%, not more than about 85%, not more than about 84%, not more than about 83%, not more than about 82%, not more than about 81%, not more than about 80%, not more than about 79%, not more than about 78%, not more than about 77%, not more than about 76%, not more than about 75%, not more than about 74%, not more than about 73%, not more than about 72%, not more than about 71%, not more than about 70%, not more than about 69%, not more than about 68%, not more than about 67%, not more than about 66%, not more than about 65%, not more than about 64%, not more than about 63%, not more than about 62%, not more than about 61%, not more than about 60%, not more than about 59%, not more than about 58%, not more than about 57%, not more than about 56%, not more than about 55%, not more than about 54%, not more than about 53%, not more than about 52%, not more than about 51%, not more than about 50%, not more than about 49%, not more than about 48%, not more than about 47%, not more than about 46%, not more than about 45%, not more than about 44%, not more than about 43%, not more than about 42%, not more than about 41%, not more than about 40%, not more than about 39%, not more than about 38%, not more than about 37%, not more than about 36%, not more than about 35%, not more than about 34%, not more than about 33%, not more than about 32%, not more than about 31%, not more than about 30%, not more than about 29%, not more than about 28%, not more than about 27%, not more than about 26%, not more than about 25%, not more than about 24%, not more than about 23%, not more than about 22%, not more than about 21%, not more than about 20%, not more than about 19%, not more than about 18%, not more than about 17%, not more than about 16%, not more than about 15%, not more than about 14%, not more than about 13%, not more than about 12%, not more than about 11%, not more than about 10%, not more than about 9%, not more than about 8%, not more than about 7%, not more than about 6%, not more than about 5%, or less than 5% relative to the comparative subject group. In some embodiments, the adverse event comprises, consists essentially of, or consists of any untoward medical occurrence in a clinical study participant (e.g., a participant in a study generally consistent with Example 11 or Example 12 described in more detail below), temporally associated with the use of study intervention, whether or not considered related to the study intervention. In some embodiments, the adverse event comprises any adverse events that occurs on or after onset of the therapeutic method (for the subject or for the subjects in the tested subject group), or on or after the onset of the comparative therapeutic method or no therapeutic method (for the comparative subject or for the subjects in the comparative subject group). In some embodiments, the adverse event comprises any adverse events that occurs on or after onset of the therapeutic method (for the subject or for the subjects in the tested subject group), or on or after the onset of the comparative therapeutic method or no therapeutic method (for the comparative subject or for the subjects in the comparative subject group), and within 30 days of the subject receiving the last dose of the therapeutic method (for the subject or for the subjects in the tested subject group), or within 30 days of the comparative therapeutic receiving the last dose of the comparative therapeutic method or cessation of no therapeutic method (for the comparative subject or for the subjects in the comparative subject group).

In some embodiments, a method of treatment consistent with the present disclosure results in a smaller (e.g., substantially smaller) portion (e.g., fraction or percentage) of subjects in a treated subject group discontinuing the method of treatment (e.g., during a clinical study generally consistent with Example 11 or Example 12) after experiencing an adverse event relative to subjects in a comparative subject group that are not treated by the same method (e.g., is treated by an antiviral monotherapy, by a FKBP ligand monotherapy, by a placebo, or receives no therapy). In some embodiments, the smaller portion is not more than about 95% relative to the comparative subject group, for example not more than about 95%, not more than about 94%, not more than about 93%, not more than about 92%, not more than about 91%, not more than about 90%, not more than about 89%, not more than about 88%, not more than about 87%, not more than about 86%, not more than about 85%, not more than about 84%, not more than about 83%, not more than about 82%, not more than about 81%, not more than about 80%, not more than about 79%, not more than about 78%, not more than about 77%, not more than about 76%, not more than about 75%, not more than about 74%, not more than about 73%, not more than about 72%, not more than about 71%, not more than about 70%, not more than about 69%, not more than about 68%, not more than about 67%, not more than about 66%, not more than about 65%, not more than about 64%, not more than about 63%, not more than about 62%, not more than about 61%, not more than about 60%, not more than about 59%, not more than about 58%, not more than about 57%, not more than about 56%, not more than about 55%, not more than about 54%, not more than about 53%, not more than about 52%, not more than about 51%, not more than about 50%, not more than about 49%, not more than about 48%, not more than about 47%, not more than about 46%, not more than about 45%, not more than about 44%, not more than about 43%, not more than about 42%, not more than about 41%, not more than about 40%, not more than about 39%, not more than about 38%, not more than about 37%, not more than about 36%, not more than about 35%, not more than about 34%, not more than about 33%, not more than about 32%, not more than about 31%, not more than about 30%, not more than about 29%, not more than about 28%, not more than about 27%, not more than about 26%, not more than about 25%, not more than about 24%, not more than about 23%, not more than about 22%, not more than about 21%, not more than about 20%, not more than about 19%, not more than about 18%, not more than about 17%, not more than about 16%, not more than about 15%, not more than about 14%, not more than about 13%, not more than about 12%, not more than about 11%, not more than about 10%, not more than about 9%, not more than about 8%, not more than about 7%, not more than about 6%, not more than about 5%, or less than 5% relative to the comparative subject group. In some embodiments, the adverse event comprises, consists essentially of, or consists of any untoward medical occurrence in a clinical study participant (e.g., a participant in a study generally consistent with Example 11 or Example 12 described in more detail below), temporally associated with the use of study intervention, whether or not considered related to the study intervention.

In some embodiments, a method of treatment consistent with the present disclosure results in a reduced (e.g., substantially reduced) time to sustained resolution or improvement of a coronavirus-related sign or symptom relative to a comparative subject or to subjects in a comparative subject group that are not treated by the same method (e.g., is treated by an antiviral monotherapy, by a FKBP ligand monotherapy, by a placebo, or receives no therapy). In some embodiments, the reduction is at least about 5%, for example at least about 5% relative to the comparative subject or to the comparative subject group, for example at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater than 99% relative to the comparative subject or to the comparative subject group. In some embodiments, the time is measured in days.

In some embodiments, a method of treatment consistent with the present disclosure results in a reduced (e.g., substantially reduced) clinical progression scale score for a subject or a subject group relative to a comparative subject or to subjects in a comparative subject group that are not treated by the same method (e.g., is treated by an antiviral monotherapy, by a FKBP ligand monotherapy, by a placebo, or receives no therapy). In some embodiments, the reduction is at least one scale score, for example at least one scale score, two scale scores, three scale scores, four scale scores, five scale scores, six scale scores, seven scale scores, eight scale scores, nine scale scores. In some embodiments, the clinical progression scale is the World Health Organization Clinical Progression Scale, for example as published online by the WHO Working Group on the Clinical Characterization and Management of COVID-19 Infection, Lancet Infect. Dis., (Jun. 12, 2020), reproduced in FIG. 30.

In some embodiments, a method of treatment consistent with the present disclosure results in a reduced (e.g., substantially reduced) time to sustained alleviation of one or more coronavirus-related symptom or all coronavirus-related symptoms relative to a comparative subject or to subjects in a comparative subject group that are not treated by the same method (e.g., is treated by an antiviral monotherapy, by a FKBP ligand monotherapy, by a placebo, or receives no therapy). In some embodiments, the reduction is at least about 5%, for example at least about 5% relative to the comparative subject or to the comparative subject group, for example at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater than 99% relative to the comparative subject or to the comparative subject group. In some embodiments, the time is measured in days. In some embodiments, the alleviation of the coronavirus-related symptom comprises assigning a severity score of 0 or 1 to the symptom, for example on a scale of 0 (no noticeable symptom) to 3 (severe symptom). In some embodiments, the alleviation of the coronavirus-related symptom comprises alleviation enduring for at least 4 days, for example enduring for at least 4 days, for at least 5 days, for at least 6 days, for at least 7 days, for at least 8 days, for at least 9 days, for at least 10 days, for at least 11 days, for at least 12 days, for at least 13 days, for at least 14 days, for at least 15 days, for at least 16 days, for at least 17 days, for at least 18 days, for at least 19 days, for at least 20 days, for at least 21 days, for at least 22 days, for at least 23 days, for at least 24 days, for at least 25 days, for at least 26 days, for at least 27 days, for at least 28 days, or for more than 28 days. In some embodiments, the assignment of the severity score is performed by the subject.

In some embodiments, a method of treatment consistent with the present disclosure results in a reduced (e.g., substantially reduced) time to an improvement (e.g., substantially improvement) of a coronavirus-related symptom relative to a comparative subject or to subjects in a comparative subject group that are not treated by the same method (e.g., is treated by an antiviral monotherapy, by a FKBP ligand monotherapy, by a placebo, or receives no therapy). In some embodiments, the reduction is at least about 5%, for example at least about 5% relative to the comparative subject or to the comparative subject group, for example at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater than 99% relative to the comparative subject or to the comparative subject group. In some embodiments, the time is measured in days. In some embodiments, the symptom is selected from the group consisting of: stuffy nose, runny nose, sore throat, shortness of breath, cough, lack of energy, tiredness, muscle aches, body aches, headache, chills, shivering, feeling hot, feeling feverish, nausea, vomiting, and diarrhea. In some embodiments the improvement comprises observing a reduction in a severity score associated with the symptom of at least 1 grade (e.g., on a 4-level scale system) compared to a baseline severity score associated with onset or peak severity of the symptom.

In some embodiments, a method of treatment consistent with the present disclosure results in subjects in a treated subject group exhibiting a reduced rate of developing severe COVID-19 relative to subjects in a comparative subject group that are not treated by the same method (e.g., is treated by an antiviral monotherapy, by a FKBP ligand monotherapy, by a placebo, or receives no therapy). In some embodiments, the reduced rate is at least about 5% relative to the comparative subject group, for example at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater than 99% relative to the comparative subject group. In some embodiments, the severe coronavirus disease state comprises, consists essentially of, or consists of assigning the subject(s) a clinical progression scale score associated with severe coronavirus disease state. In some embodiments the clinical progression scale score is a WHO Clinical Progression Scale score of 6, 7, 8, 9, or 10. In some embodiments, the severe coronavirus disease state comprises of one or more of: (i) observing a resting oxygen saturation level in the subject(s) of less than 94%, (ii) observing hospitalization of the subject(s) for any reason, and/or (iii) death of the subject(s).

In some embodiments, a method of treatment consistent with the present disclosure results in subjects in a treated subject group exhibiting a reduced rate of death relative to subjects in a comparative subject group that are not treated by the same method (e.g., is treated by an antiviral monotherapy, by a FKBP ligand monotherapy, by a placebo, or receives no therapy). In some embodiments, the reduced rate is at least about 5% relative to the comparative subject group, for example at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater than 99% relative to the comparative subject group. In some embodiments, the death is from any cause. In other embodiments, the death results from a coronavirus-associated complication.

In some embodiments, a method of treatment consistent with the present disclosure results in subjects in a treated subject group exhibiting a reduced hospitalization rate relative to subjects in a comparative subject group that are not treated by the same method (e.g., is treated by an antiviral monotherapy, by a FKBP ligand monotherapy, by a placebo, or receives no therapy). In some embodiments, the reduced rate is at least about 5% relative to the comparative subject group, for example at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater than 99% relative to the comparative subject group. In some embodiments, the hospitalization is from any cause. In other embodiments, the hospitalization results from a coronavirus-associated complication. In some embodiments, the coronavirus-associated complication is respiratory distress. In some embodiments, the coronavirus-associated complication is low O₂ saturation, such as an O₂ saturation level of less than 93%.

In some embodiments, a method of treatment consistent with the present disclosure results in a reduced (e.g., substantially reduced) time from onset of therapy to determination of no detectable coronavirus RNA in a subject or in subjects in a treated subject group, relative to a comparative subject or to subjects in a comparative subject group that are not treated by the same method (e.g., is treated by an antiviral monotherapy, by a FKBP ligand monotherapy, by a placebo, or receives no therapy). In some embodiments, the reduction is at least about 5%, for example at least about 5% relative to the comparative subject or to the comparative subject group, for example at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater than 99% relative to the comparative subject or to the comparative subject group. In some embodiments, the time is measured in days. In some embodiments, the viral RNA is measured in saliva of the subject(s). In some embodiments, determining viral RNA presence and/or amount comprises performing a PCR assay (e.g., a RT-PCR assay) on a sample obtained from the subject(s). In some such embodiments, the sample comprises, consists essentially of, or consists of saliva.

In some embodiments, a method of treatment consistent with the present disclosure results in a subject or in subjects in a treated subject group exhibiting a greater improvement in clinical status after a period of time from onset of treatment relative to subjects in a comparative subject group that are not treated by the same method (e.g., is treated by an antiviral monotherapy, by a FKBP ligand monotherapy, by a placebo, or receives no therapy). In some embodiments, the improvement is at least about 5% relative to the comparative subject group, for example at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater than 99% relative to the comparative subject group. In some embodiments, the severe coronavirus disease state comprises, consists essentially of, or consists of assigning the subject(s) a clinical status scale score at the onset of the therapeutic method (for the subject or for the subjects in the treated subject group) or at the onset of the comparative method (for subjects in the comparative subject group receiving a different therapy or no therapy). In some embodiments, the clinical status scale is the World Health Organization Clinical Progression Scale. In some embodiments the clinical status scale score comprises, consists essentially of, or consists of an ordinal scale, such as:
1: death;
2: hospitalized, receiving invasive mechanical ventilation or extracorporeal membrane oxygen (“ECMO”);
3: hospitalized, receiving noninvasive ventilation or high-flow oxygen devices;
4: hospitalized, requiring low-flow supplemental oxygen;
5: hospitalized, not requiring supplemental oxygen but receiving ongoing medical care (related or not related to coronavirus);
6: hospitalized, requiring neither supplemental oxygen nor ongoing medical care; and
7: not hospitalized.

In some embodiments, a method of treatment consistent with the present disclosure results in a reduced (e.g., substantially reduced) time to improvement (e.g., substantial improvement) of clinical status of a subject or subject in a tested subject group relative to a comparative subject or to subjects in a comparative subject group that are not treated by the same method (e.g., is treated by an antiviral monotherapy, by a FKBP ligand monotherapy, by a placebo, or receives no therapy). In some embodiments, the reduction is at least about 5%, for example at least about 5% relative to the comparative subject or to the comparative subject group, for example at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater than 99% relative to the comparative subject or to the comparative subject group. In some embodiments, the time is measured in days. In some embodiments, the method comprises assigning an initial clinical status scale score to the subject or to each subject in the treated subject group, and assigning an initial clinical status scale score to the comparative subject or to each subject in the comparative subject group; and observing an improved clinical status scale score in the subject or in each subject in the tested subject group and an improved clinical status scale score in the comparative subject or in each subject in the comparative subject group. In some embodiments, the improved clinical status scale score comprises an improvement of at least two levels of the clinical status scale score. In some embodiments, the clinical status scale is the World Health Organization Clinical Progression Scale. In some embodiments the clinical status scale score comprises, consists essentially of, or consists of an ordinal scale, such as:
1: death;
2: hospitalized, receiving invasive mechanical ventilation or ECMO;
3: hospitalized, receiving noninvasive ventilation or high-flow oxygen devices;
4: hospitalized, requiring low-flow supplemental oxygen;
5: hospitalized, not requiring supplemental oxygen but receiving ongoing medical care (related or not related to coronavirus);
6: hospitalized, requiring neither supplemental oxygen nor ongoing medical care; and
7: not hospitalized.

In some embodiments, a method of treatment consistent with the present disclosure results in a reduced (e.g., substantially reduced) time to recovery (e.g., substantial recovery) of a subject or subject in a tested subject group relative to a comparative subject or to subjects in a comparative subject group that are not treated by the same method (e.g., is treated by an antiviral monotherapy, by a FKBP ligand monotherapy, by a placebo, or receives no therapy).

In some embodiments, the reduction is at least about 5%, for example at least about 5% relative to the comparative subject or to the comparative subject group, for example at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater than 99% relative to the comparative subject or to the comparative subject group. In some embodiments, the time is measured in days. In some embodiments, the recovery comprises observing in the subject or in each subject in the tested subject group and/or in each subject in the comparative subject group a clinical status of (i) hospitalized but requiring neither supplemental oxygen nor ongoing medical care, or (ii) not hospitalized. In some embodiments, the method comprises comparing (a) a time (e.g., average time) from onset of therapy (for the subject or for the subjects in the tested subject group) to recovery, and (b) a time (e.g., average time) from onset of comparative therapy (for the comparative subject or for the subjects in the comparative subject group) to recovery. In some embodiments, determining a status of recovery in the subject comprises determining that the subject is well enough for hospital discharge or to return to a normal activity level. In some embodiments, the method comprises assigning an initial clinical status scale score to the subject or to each subject in the treated subject group, and assigning an initial clinical status scale score to the comparative subject or to each subject in the comparative subject group; and observing an improved clinical status scale score in the subject or in each subject in the tested subject group and an improved clinical status scale score in the comparative subject or in each subject in the comparative subject group. In some embodiments, the initial clinical status scale score corresponds to a determination that the subject(s) or comparative subject(s) is (i) hospitalized and receiving invasive mechanical ventilation or ECMO, (ii) hospitalized and receiving noninvasive ventilation or high-flow oxygen devices, (iii) hospitalized and requiring low-flow supplemental oxygen, or (iv) hospitalized and not requiring supplemental oxygen but receiving ongoing medical care related or not related to coronavirus. In some embodiments, the improved clinical status scale score corresponds to a determination that the subject(s) or comparative subject(s) is (i) hospitalized but requiring neither supplemental oxygen nor ongoing medical care, or (ii) not hospitalized. In some embodiments, the improved clinical status scale score comprises an improvement of at least two levels of the clinical status scale score. In some embodiments, the clinical status scale is the World Health Organization Clinical Progression Scale. In some embodiments the clinical status scale score comprises, consists essentially of, or consists of an ordinal scale, such as:
1: death;
2: hospitalized, receiving invasive mechanical ventilation or ECMO;
3: hospitalized, receiving noninvasive ventilation or high-flow oxygen devices;
4: hospitalized, requiring low-flow supplemental oxygen;
5: hospitalized, not requiring supplemental oxygen but receiving ongoing medical care (related or not related to coronavirus);
6: hospitalized, requiring neither supplemental oxygen nor ongoing medical care; and
7: not hospitalized.

In some embodiments, a method of treatment consistent with the present disclosure results in a reduced (e.g., substantially reduced) time to modified recovery (e.g., partial recovery) of a subject or subject in a tested subject group relative to a comparative subject or to subjects in a comparative subject group that are not treated by the same method (e.g., is treated by an antiviral monotherapy, by a FKBP ligand monotherapy, by a placebo, or receives no therapy). In some embodiments, the reduction is at least about 5%, for example at least about 5% relative to the comparative subject or to the comparative subject group, for example at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater than 99% relative to the comparative subject or to the comparative subject group. In some embodiments, the time is measured in days. In some embodiments, the modified recovery comprises observing in the subject or in each subject in the tested subject group and/or in each subject in the comparative subject group an improved clinical status from:
an initial clinical status of:
   (a) hospitalized and receiving invasive mechanical ventilation or ECMO;
   (b) hospitalized and receiving noninvasive ventilation or high-flow oxygen devices; or
   (c) hospitalized and requiring low-flow supplemental oxygen
to a modified recovery clinical status of:
   (i) hospitalized but not requiring supplemental oxygen but receiving ongoing medical care (related or not related to coronavirus);
   (ii) hospitalized but requiring neither supplemental oxygen nor ongoing medical care; or
   (iii) not hospitalized.

In some embodiments, the modified recovery comprises observing in the subject or in each subject in the tested subject group and/or in each subject in the comparative subject group an improved clinical status from:
an initial clinical status of hospitalized but not requiring supplemental oxygen but receiving ongoing medical care (related or not related to coronavirus)
to a modified recovery clinical status of:
   (i) hospitalized but requiring neither supplemental oxygen nor ongoing medical care; or
   (ii) not hospitalized.

In some embodiments, the method comprises comparing (a) a time (e.g., average time) from onset of therapy (for the subject or for the subjects in the tested subject group) to modified recovery, and (b) a time (e.g., average time) from onset of comparative therapy (for the comparative subject or for the subjects in the comparative subject group) to modified recovery. In some embodiments, the method comprises assigning an initial clinical status scale score to the subject or to each subject in the treated subject group, and assigning an initial clinical status scale score to the comparative subject or to each subject in the comparative subject group; and observing an improved clinical status scale score in the subject or in each subject in the tested subject group and an improved clinical status scale score in the comparative subject or in each subject in the comparative subject group. In some embodiments, the improved clinical status scale score comprises an improvement of at least two levels of the clinical status scale score. In some embodiments, the clinical status scale is the World Health Organization Clinical Progression Scale. In some embodiments the clinical status scale score comprises, consists essentially of, or consists of an ordinal scale, such as:
1: death;
2: hospitalized, receiving invasive mechanical ventilation or ECMO;
3: hospitalized, receiving noninvasive ventilation or high-flow oxygen devices;
4: hospitalized, requiring low-flow supplemental oxygen;
5: hospitalized, not requiring supplemental oxygen but receiving ongoing medical care (related or not related to coronavirus);
6: hospitalized, requiring neither supplemental oxygen nor ongoing medical care; and
7: not hospitalized.

In some embodiments, a method of treatment consistent with the present disclosure results in subjects in a treated subject group exhibiting a reduced rate of thromboembolism relative to subjects in a comparative subject group that are not treated by the same method (e.g., is treated by an antiviral monotherapy, by a FKBP ligand monotherapy, by a placebo, or receives no therapy). In some embodiments, the reduced rate is at least about 5% relative to the comparative subject group, for example at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or greater than 99% relative to the comparative subject group. In some embodiments, the thromboembolism is from any cause. In other embodiments, the thromboembolism results from a coronavirus-associated complication. In some embodiments, the coronavirus-associated complication is respiratory distress. In some embodiments, the coronavirus-associated complication is low O₂ saturation, such as an O₂ saturation level of less than 93%.

The term “treat” or “treatment” as used herein refers to any treatment of a disorder or disease, and includes, but is not limited to, preventing the disorder or disease from occurring in a subject that may be predisposed to the disorder or disease but has not yet been diagnosed as having the disorder or disease; inhibiting the disorder or disease, for example, arresting the development of the disorder or disease; relieving the disorder or disease, for example, causing regression of the disorder or disease; or relieving the condition caused by the disease or disorder, for example, stopping the symptoms of the disease or disorder.

The term “prevent” or “prevention,” in relation to a disorder or disease, means preventing the onset of disorder or disease development if none had occurred, or preventing further symptoms or disease development if the disorder or disease was already present.

The term “co-administration” as used herein refers to, for example, administration of two or more agents (e.g., an FKBP ligand or a derivative thereof and an antiviral agent) to the subject at the same time or one agent after the other, such as one immediately after the other, within five minutes of each other, within ten minutes of each other, within fifteen minutes of each other, within thirty minutes of each other, within one hour of each other, within two hours of each other, within four hours of each other, within six hours of each other, within twelve hours of each other, within one day of each other, within one week of each other, within two weeks of each other, within one month of each other, within two months of each other, within six months of each other, within one year of each other, etc. Co-administration of the FKBP ligand or derivative thereof and the antiviral agent may occur by administration of a single dosage unit (e.g., a single oral dosage unit or a single parenteral injection solution) that includes both the FKBP ligand or derivative thereof and the antiviral agent. In such embodiments, the term “co-administration” generally refers to administration of the FKBP ligand or derivative thereof and the antiviral agent to the subject at the same time. In embodiments wherein the FKBP ligand or derivative thereof and the antiviral agent are housed in separate dosage units (e.g., in separate oral dosage units, in separate parenteral injection solutions, or one in an oral dosage unit and the other in a parental injection solution), co-administration of the FKBP ligand or derivative thereof and the antiviral agent to the subject may occur at the same time or one agent after the other, such as one immediately after the other, within five minutes of each other, within ten minutes of each other, within fifteen minutes of each other, within thirty minutes of each other, within one hour of each other, within two hours of each other, within four hours of each other, within six hours of each other, within twelve hours of each other, within one day of each other, within one week of each other, within two weeks of each other, within one month of each other, within two months of each other, within six months of each other, within one year of each other, etc.

The term “subject in need thereof” refers generally to a subject that already has a coronavirus infection (whether diagnosed or undiagnosed), to a subject that is at elevated risk of developing a coronavirus infection, and to a subject that previously had a coronavirus infection (whether previously diagnosed or never diagnosed) but does not exhibit antibodies (e.g., sufficient levels of antibodies) to ward of a subsequent coronavirus infection.

### EXAMPLES

Aspects of embodiments may be further understood in light of the following examples, which should not be construed as limiting in any way.

### Example 1

Potential non-immunosuppressive FKBP ligands will be tested in vitro for antiviral activity against SARS Cov2 to select one or more lead compounds. Based on the results of these in-vitro studies, in vivo animal testing will be performed on at least one of the lead compounds. In parallel with the in vivo animal testing, IND enabling pre-clinical ADME, Safety pharmacology and toxicology testing will be performed if not already completed. Initial clinical testing will be in normal volunteers (e.g., subjects without known coronavirus infections), and will include single dose and multiple dose evaluation of pharmacokinetics, safety and tolerability aspects if not already completed. After completion of the normal volunteer studies, Phase 2 studies of subjects infected with SARS-COV2 will be initiated.

### Example 2

The BSL-3 model is a cell-based model using replication competent (live) SARS-Cov2. Vero76 cells are infected with the live virus at low MOI and multiple rounds of viral replication occur over the course of the assay (approximately day 6 endpoint). Virally infected, untreated cells are compared to virally infected cells, and cytopathic effects are quantified by neutral red endpoint. Essentially, viral infection kills the cells, and assessment is made of the differential between the treated and untreated cells. Based on these data, an 8-point dose response curve of test article is created in triplicate, with toxicity assessed in parallel. Toxicity will be assessed by comparing untreated, uninfected cells with compound-treated cells. A QC control is included with each assess run to ensure the assay is performing as expected.

If in vitro activity against SARS COV 2 is confirmed, further in vitro work will proceed to test combinations with other antivirals and activity against other coronaviruses.

### Example 3

Ferrets will be treated with placebo or FKBP ligand and will be challenged with approximately 1×10⁶ TCID₅₀ of SARS-COV-2 on day 0 via the intranasal route. Animals will be monitored twice daily, at a minimum and at least 6 hours apart, for signs of illness or distress, for a period of 14 days post-challenge. Animals will then be anesthetized and nasal wash samples collected. Nasal washes will be assayed for virus concentration by TCID₅₀ for live virus and by SARS-CoV-2-specific RT-qPCR. Additionally, tissue viral burden by TCID₅₀ and/or RT-qPCR will be measured and the histopathology of various organs and tissues will be assessed.

### Example 4

The mouse-adapted model of SARS-COV-2 (e.g., K. H. Dinnon, et al., Nature, vol. 586, pp. 560-66 (2020)) and/or the human ACE2 transduced/transgenic mouse model (e.g., J. Sun, et al., Cell, vol. 182(3), pp. 734-43 (2020)) will be used to assess the efficacy of an FKBP ligand in combination with an anti-viral agent consistent with the present disclosure. Mice will be treated with placebo, FKBP ligand alone, an antiviral agent alone, or the combination of FKBP and anti-viral agent at various doses after infection with SARS-COV-2. Animals will be monitored daily. Mortality, lung virus titers and body weight will be assessed.

### Example 5

The golden Syrian Hamster model (e.g., M. Imai, et al., Proc. Nat'l Acad. Sci. (USA), vol. 117(28), pp. 16587-95 (2020)) will be used to assess the efficacy of an FKBP ligand in combination with an anti-viral agent consistent with the present disclosure. Hamsters will be inoculated intranasally with SARS-COV-2. After inoculation, treatment will be initiated with placebo, FKBP ligand alone, an antiviral agent alone, or the combination of FKBP ligand with an antiviral agent at various doses. Animals will be monitored daily. Mortality, viral RNA loads in the lungs, and body weight will be assessed.

### Example 6

The synergistic therapeutic effects of FK1706 with either remdesivir or M128533 were determined in an in vitro anti-SARS COV-2 cytoprotection assay.

Test compounds were serially diluted using four dilutions in test medium (MEM supplemented with 2% FBS and 50 μg/ml gentamicin). Each dilution was added to 5 wells of a 96-well plate with 80-100% confluent Vero E6 cells. Three wells of each dilution were infected with SARS CoV-2 virus, and two wells remained uninfected as toxicity controls. Six wells were infected and untreated as virus controls, and six wells were uninfected and untreated as cell controls. Viruses were prepared to achieve the lowest possible multiplicity of infection (MOI) that would yield >80% cytopathic effect (CPE) at 4 days.

M128533 was tested in parallel as a positive control. Plates were incubated at 37±2° C., 5% CO₂. For neutral red assay, on day 4 post-infection, once untreated virus control wells reached maximum CPE, plates were stained with neutral red dye for approximately 2 hours (+15 minutes). Supernatant dye was removed, wells were rinsed with PBS, and the incorporated dye was extracted in 50:50 Sorensen citrate buffer/ethanol for >30 minutes and the optical density was read on a spectrophotometer at 540 nm. Optical densities were converted to percent of cell controls and normalized to the virus control, then the concentration of test compound required to inhibit CPE by 50% (EC₅₀) was calculated by regression analysis. The concentration of compound that would cause 50% cell death in the absence of virus was similarly calculated (TC₅₀). The selective index (TI) is the TC₅₀ divided by EC₅₀.

Results are summarized in Table 1.

TABLE 1
Anti-SARS-CoV2 Cytoprotection Assay Results

| | VeroE6/SARS CoV-2 (USA-WA1/2020) | | | | | |
|---|---|---|---|---|---|---|
| Compound(s) | EC₅₀ (μM) | | TC₅₀ (μM) | | TI | |
| FK1706 | >90 | >90 | >90 | >90 | — | — |

| | | | |
|---|---|---|---|
| FK1706 + Remdesivir (3 μM) | <11.3 | >90 | >7.96 |
| Remdesivir (single conc.) | >3 | >3 | — |
| Remdesivir | 3.63 | >100 | >27.5 |
| FK1706 + M128533 (1 | <11.3 | >90 | >7.96 |
| μg/mL) | | | |
| M128533 (single conc.) | >1 | >1 | — |
| M128533 (μg/mL) | 1.53 | 86.8 | 56.7 |

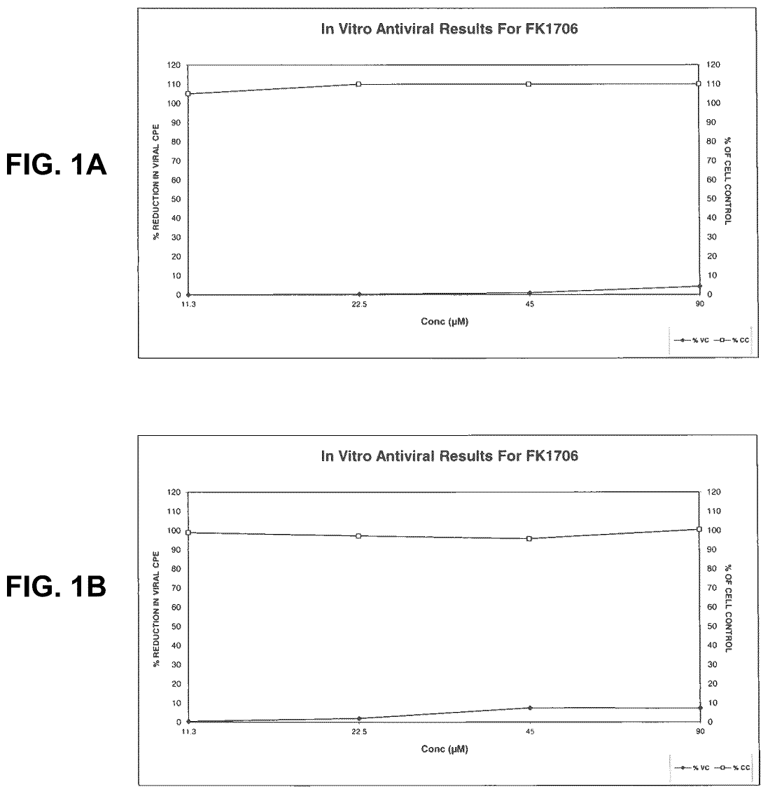

Reduction in viral CPE and cell control for FK1706 alone, as a function of FK1706 concentration, are shown graphically in FIGS. 1A-1B (two trials).

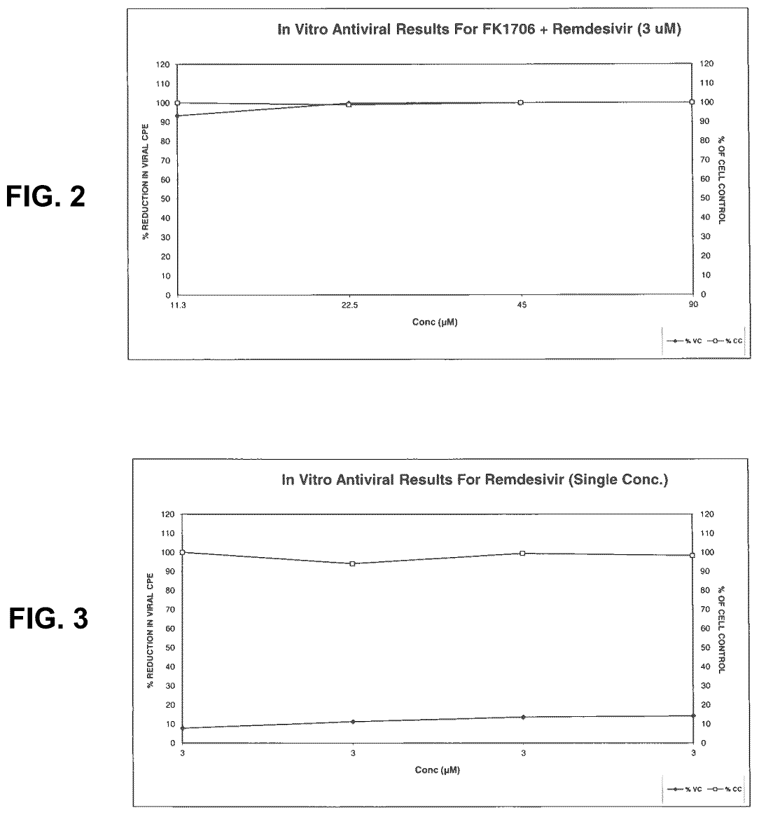

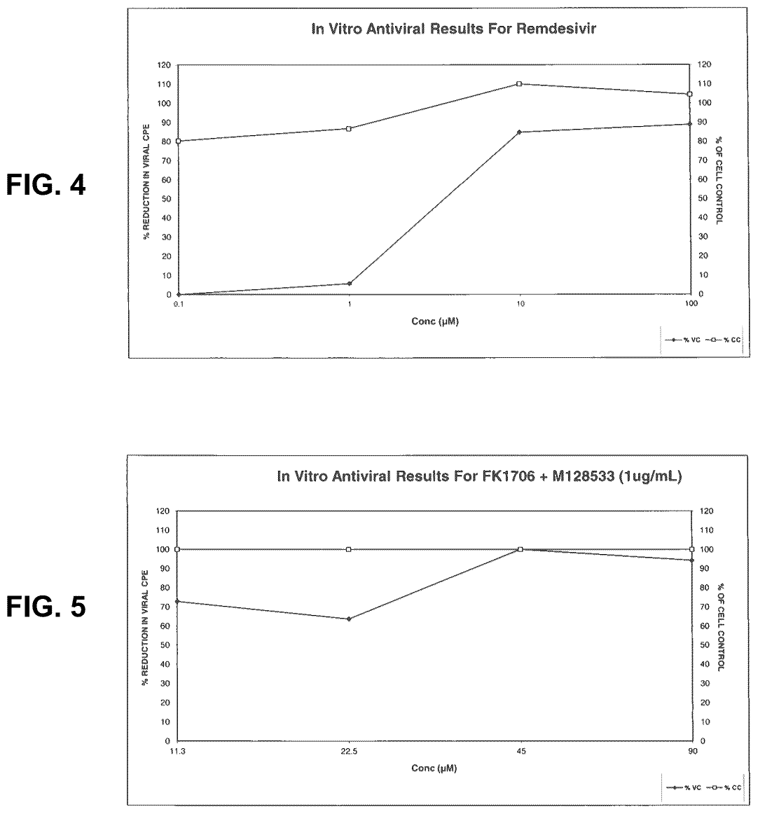

Synergy between FK1706 and sub-therapeutic Remdesivir (3 μM) is shown graphically in FIG. 2. Antiviral activities of sub-therapeutic Remdesivir (3 μM) and as a function of Remdesivir concentration (0.1 to 100 μM) are shown in FIGS. 3-4 for comparison. Alone, FK1706 showed little reduction in SARS COV-2 virus (0-7%) across the range of 11-90 μM. Remdesivir administered alone at sub-therapeutic levels (e.g., 3 μM) also showed very little reduction in SARS COV-2 virus (5-14%). When co-administered, however, FK1706 (11-90 μM) and Remdesivir (3 μM) substantially or even completely reduced SARS COV-2 virus (93-100%).

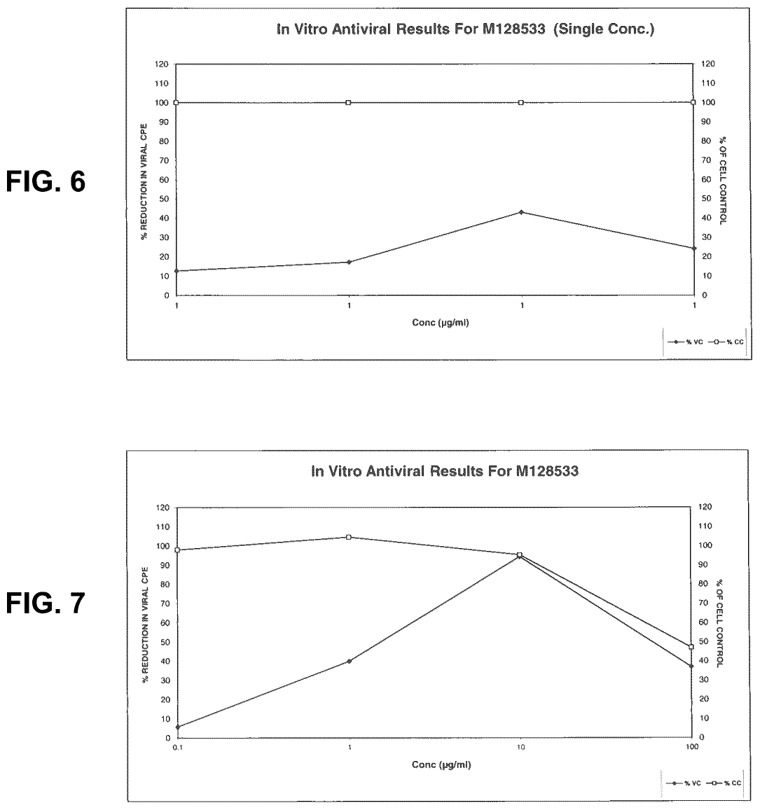

Synergy between FK1706 and sub-therapeutic M128533 (1 μg/mL) is shown graphically in FIG. 5. Antiviral activities of sub-therapeutic M128533 (1 μg/ml) and as a function of M128533 concentration (0.1 to 100 μg/ml) are shown in FIGS. 6-7 for comparison. Alone, FK1706 showed little reduction in SARS COV-2 virus (0-7%) across the range of 11-90 μM. M128533 administered alone at sub-therapeutic levels (e.g., 1 μg/mL) also showed very little reduction in SARS Cov-2 virus (12-43%). When co-administered, however, FK1706 (11-90 UM) and M128533 (1 μg/mL) substantially or even completely reduced SARS COV-2 virus (64-100%).

These data demonstrate that sub-therapeutic FK1706 combined with sub-therapeutic Remdesivir or with sub-therapeutic M128533 act synergistically to suppress SARS-COV-2 virus.

### Example 7

The synergistic therapeutic effects of ElteN378 with either remdesivir or M128533 were determined in an in vitro anti-SARS COV-2 cytoprotection assay.

Test compounds were serially diluted using four dilutions in test medium (MEM supplemented with 2% FBS and 50 μg/ml gentamicin). Each dilution was added to 5 wells of a 96-well plate with 80-100% confluent Vero E6 cells. Three wells of each dilution were infected with SARS CoV-2 virus, and two wells remained uninfected as toxicity controls. Six wells were infected and untreated as virus controls, and six wells were uninfected and untreated as cell controls. Viruses were prepared to achieve the lowest possible multiplicity of infection (MOI) that would yield >80% cytopathic effect (CPE) at 3 days.

M128533 was tested in parallel as a positive control. Plates were incubated at 37±2° C., 5% CO₂. For neutral red assay, on day 3 post-infection, once untreated virus control wells reached maximum CPE, plates were stained with neutral red dye for approximately 2 hours (+15 minutes). Supernatant dye was removed, wells were rinsed with PBS, and the incorporated dye was extracted in 50:50 Sorensen citrate buffer/ethanol for >30 minutes and the optical density was read on a spectrophotometer at 540 nm. Optical densities were converted to percent of cell controls and normalized to the virus control, then the concentration of test compound required to inhibit CPE by 50% (EC₅₀) was calculated by regression analysis. The concentration of compound that would cause 50% cell death in the absence of virus was similarly calculated (TC₅₀). The selective index (TI) is the TC₅₀ divided by EC₅₀.

Results are summarized in Table 2.

TABLE 2
Anti-SARS-CoV2 Cytoprotection Assay Results

| | VeroE6/SARS CoV-2 (USA-WA1/2020) | | |
|---|---|---|---|
| Compound(s) | EC₅₀ (μM) | TC₅₀ (μM) | TI |
| ElteN378 | >30 | >30 | — |
| ElteN378 + Remdesivir (3 | 19.6 | >30 | >1.53 |
| μM) | | | |
| Remdesivir (single conc.) | >3 | >3 | — |
| Remdesivir | 7.31 | >100 | >13.7 |
| ElteN378 + M128533 (1 | <1.11 | >30 | >27 |
| μg/mL) | | | |
| M128533 (single conc.) | — | >1 | — |
| M128533 (μg/mL) | 1.36 | >100 | >73.5 |

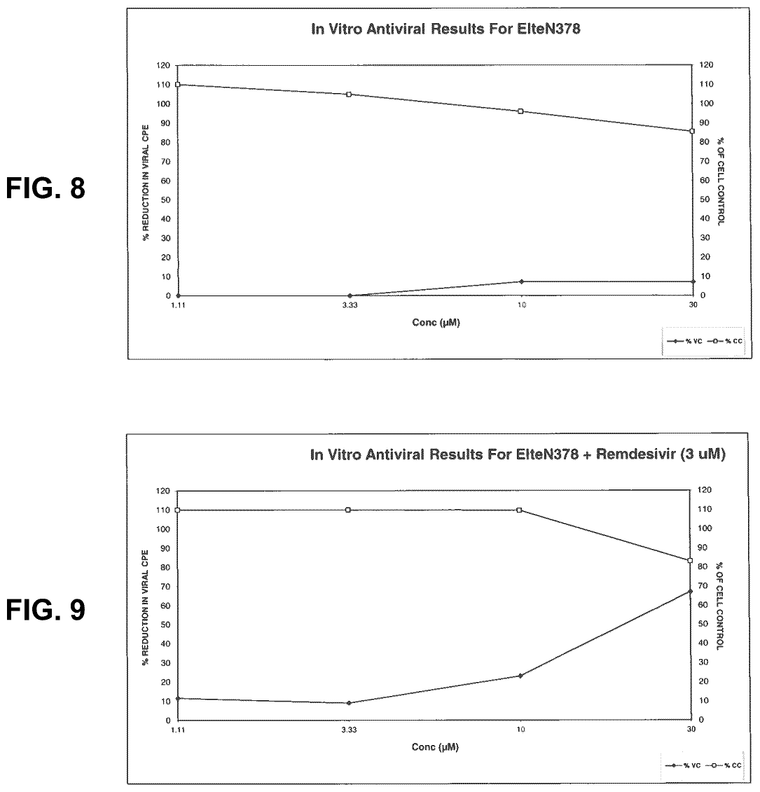

Reduction in viral CPE and cell control for ElteN378 alone, as a function of ElteN378 concentration, are shown graphically in FIG. 8.

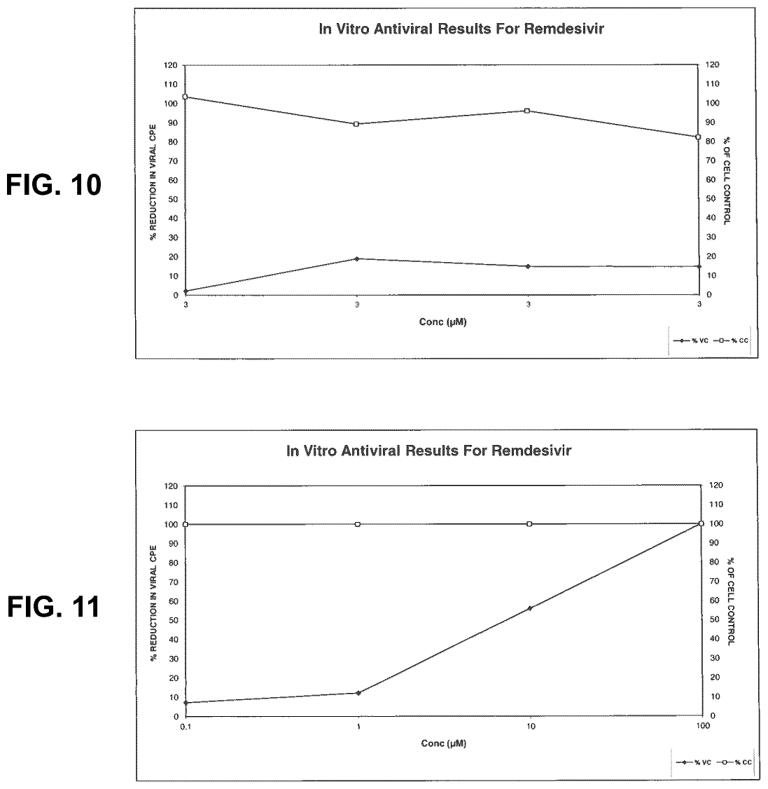

Synergy between ElteN378 and sub-therapeutic Remdesivir (3 μM) is shown graphically in FIG. 9. Antiviral activities of sub-therapeutic Remdesivir (3 μM) and as a function of Remdesivir concentration (0.1 to 100 μM) are shown in FIGS. 10-11 for comparison. Alone, ElteN378 showed little reduction in SARS COV-2 virus (0-7%) across the range of 0.12-30 μM. Remdesivir administered alone at sub-therapeutic levels (e.g., 3 μM) also showed very little reduction in SARS COV-2 virus (2-19%). When co-administered, however, ElteN378 (0.12-30 μM) and Remdesivir (3 μM) substantially reduced SARS COV-2 virus (up to 70% reduction in viral CPE at 30 μM ElteN378 with 3 μM remdesivir).

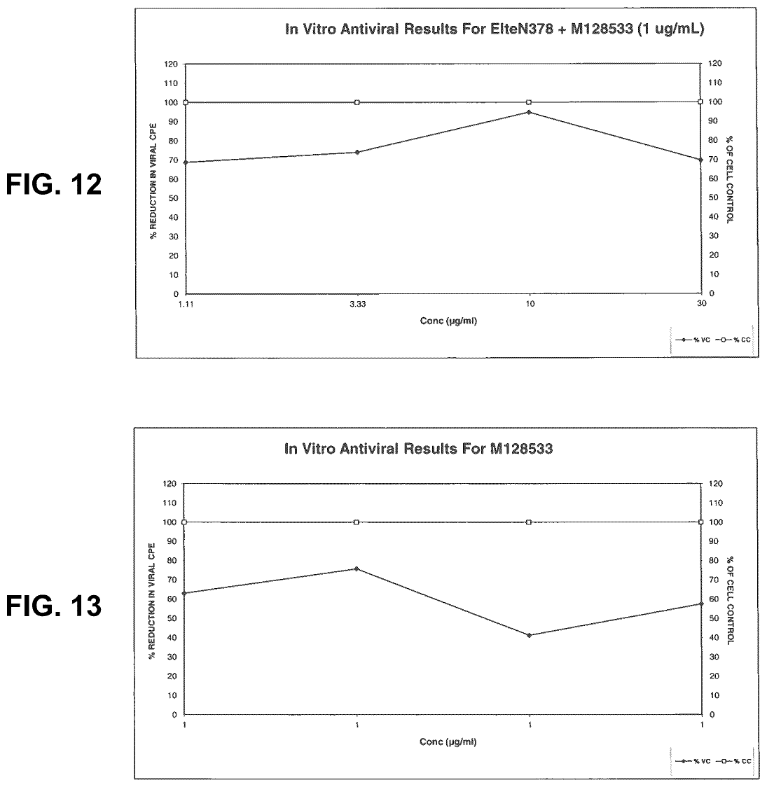

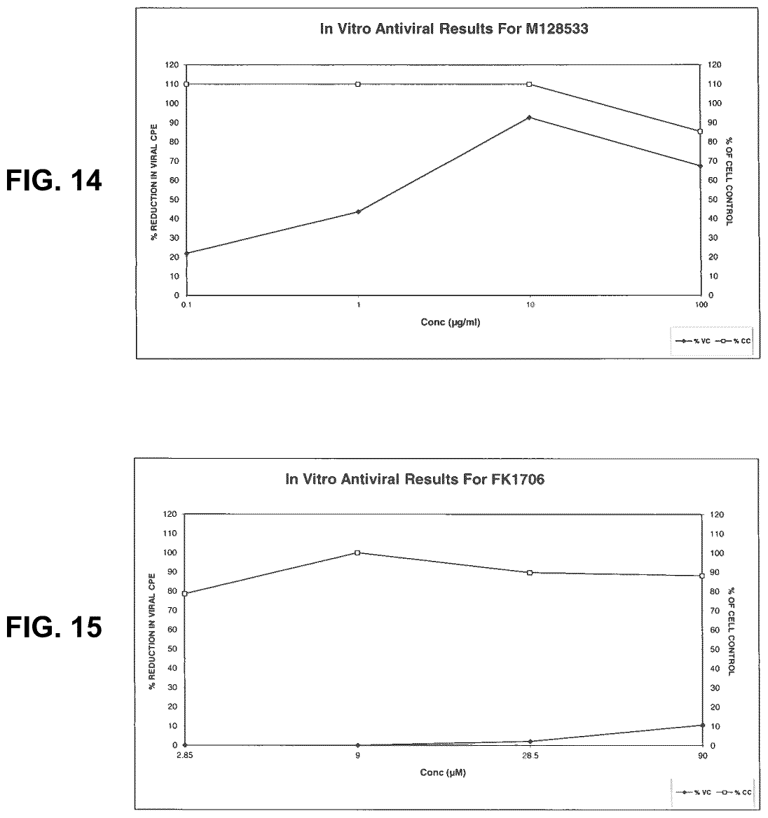

Synergy between ElteN378 and sub-therapeutic M128533 (1 μg/mL) is shown graphically in FIG. 12. Antiviral activities of sub-therapeutic M128533 (1 μg/ml) and as a function of M128533 concentration (0.1 to 100 g/ml) are shown in FIGS. 13-14 for comparison. Alone, ElteN378 showed little reduction in SARS COV-2 virus (0-7%) across the range of 0.12-30 μM. M128533 administered alone at sub-therapeutic levels (e.g., 1 μg/mL) also showed suboptimal reduction in SARS COV-2 virus (41-76%). When co-administered, however, ElteN378 (0.12-30 UM) and M128533 (1 μg/mL) substantially or even completely reduced SARS COV-2 virus (69-95%).

These data demonstrate that sub-therapeutic ElteN378 combined with sub-therapeutic remdesivir or with sub-therapeutic M128533 act synergistically to suppress SARS-COV-2 virus.

### Example 8

The synergistic therapeutic effects of FK1706 and molnupiravir were determined in an in vitro anti-SARS COV-2 cytoprotection assay.

Test compounds were serially diluted using four dilutions in test medium (MEM supplemented with 2% FBS and 50 μg/ml gentamicin). Each dilution was added to 5 wells of a 96-well plate with 80-100% confluent Vero E6 cells. Three wells of each dilution were infected with SARS CoV-2 virus, and two wells remained uninfected as toxicity controls. Six wells were infected and untreated as virus controls, and six wells were uninfected and untreated as cell controls. Viruses were prepared to achieve the lowest possible multiplicity of infection (MOI) that would yield >80% cytopathic effect (CPE) at 3 days.

Remdesivir was tested in parallel as a positive control and provided a TI value of >36. Plates were incubated at 37±2° C., 5% CO₂. For neutral red assay, on day 3 post-infection, once untreated virus control wells reached maximum CPE, plates were stained with neutral red dye for approximately 2 hours (±15 minutes). Supernatant dye was removed, wells were rinsed with PBS, and the incorporated dye was extracted in 50:50 Sorensen citrate buffer/ethanol for >30 minutes and the optical density was read on a spectrophotometer at 540 nm. Optical densities were converted to percent of cell controls and normalized to the virus control, then the concentration of test compound required to inhibit CPE by 50% (EC₅₀) was calculated by regression analysis. The concentration of compound that would cause 50% cell death in the absence of virus was similarly calculated (TC₅₀). The selective index (TI) is the TC₅₀ divided by EC₅₀.

Results are summarized in Table 3.

TABLE 3
Anti-SARS-CoV2 Cytoprotection Assay Results

| | VeroE6/SARS CoV-2 (USA-WA1/2020) | | |
|---|---|---|---|
| Compound(s) | EC₅₀ (μM) | TC₅₀ (μM) | TI |
| FK1706 | >90 | >90 | — |
| FK1706 + Molnupiravir | 28.7 | >90 | >3.14 |
| (0.3 μM) | | | |
| Molnupiravir (single conc.) | >0.3 | >0.3 | — |

Reduction in viral CPE (solid circles) and cell control (open circles) for FK1706 alone, as a function of FK1706 concentration, are shown graphically in FIG. 15.

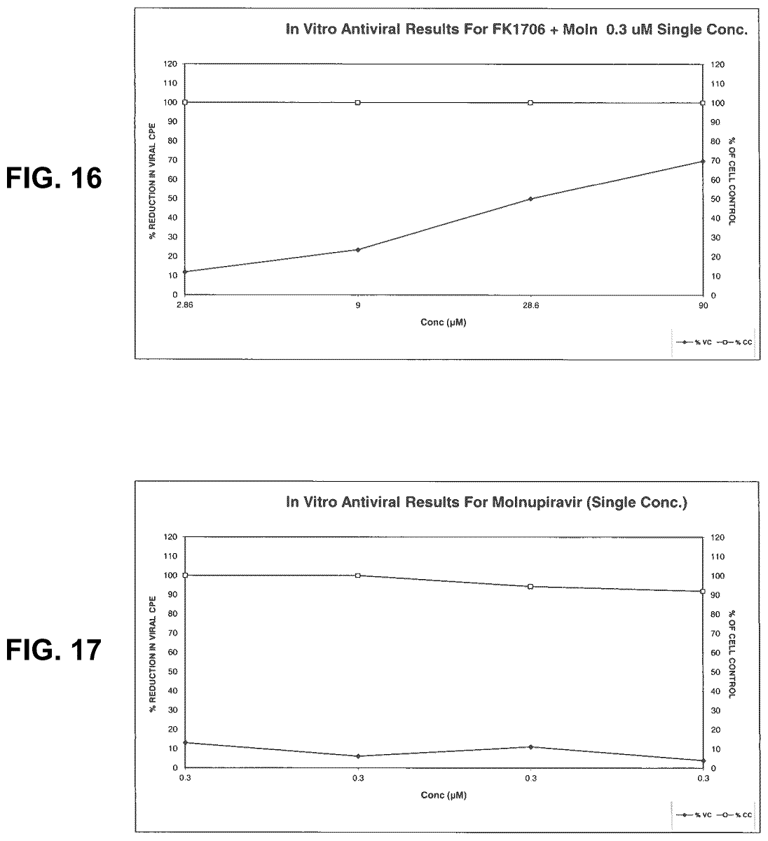

Synergy between FK1706 and sub-therapeutic molnupiravir (0.3 μM) is shown graphically in FIG. 16. Antiviral activity of sub-therapeutic molnupiravir (0.3 μM) is shown in FIG. 17 for comparison. Alone, FK1706 showed little reduction in SARS COV-2 virus (0-10%) across the range of 2.85-90 μM. Molnupiravir administered alone at sub-therapeutic levels (e.g., 0.3 μM) also showed very little reduction in SARS COV-2 virus (4-13%). When co-administered, however, FK1706 (2.85-90 UM) and sub-therapeutic molnupiravir (0.3 μM) substantially reduced SARS COV-2 virus (up to 70% reduction in viral CPE at 90 M FK1706 with 0.3 UM molnupiravir).

These data demonstrate that sub-therapeutic FK1706 combined with sub-therapeutic molnupiravir act synergistically to suppress SARS-COV-2 virus.

### Example 9

The synergistic therapeutic effects of GPI-1485 and either molnupiravir or remdesivir were determined in an in vitro anti-SARS CoV-2 cytoprotection assay.

Test compounds were serially diluted using four dilutions in test medium (MEM supplemented with 2% FBS and 50 μg/ml gentamicin). Each dilution was added to 5 wells of a 96-well plate with 80-100% confluent Vero E6 cells. Three wells of each dilution were infected with SARS CoV-2 virus, and two wells remained uninfected as toxicity controls. Six wells were infected and untreated as virus controls, and six wells were uninfected and untreated as cell controls. Viruses were prepared to achieve the lowest possible multiplicity of infection (MOI) that would yield >80% cytopathic effect (CPE) at 3 days.

Remdesivir was tested in parallel as a positive control and provided a TI value of >31.3. Plates were incubated at 37±2° C., 5% CO₂. For neutral red assay, on day 3 post-infection, once untreated virus control wells reached maximum CPE, plates were stained with neutral red dye for approximately 2 hours (±15 minutes). Supernatant dye was removed, wells were rinsed with PBS, and the incorporated dye was extracted in 50:50 Sorensen citrate buffer/ethanol for >30 minutes and the optical density was read on a spectrophotometer at 540 nm. Optical densities were converted to percent of cell controls and normalized to the virus control, then the concentration of test compound required to inhibit CPE by 50% (EC₅₀) was calculated by regression analysis. The concentration of compound that would cause 50% cell death in the absence of virus was similarly calculated (TC₅₀). The selective index (TI) is the TC₅₀ divided by EC₅₀.

Results are summarized in Table 4.

TABLE 4
Anti-SARS-CoV2 Cytoprotection Assay Results

| | VeroE6/SARS CoV-2 (USA-WA1/2020) | | |
|---|---|---|---|
| Compound(s) | EC₅₀ (μM) | TC₅₀ (μM) | TI |
| GPI-1485 | >320 | >320 | — |
| GPI-1485 + Molnupiravir (0.3 | >320 | >320 | — |
| μM) | | | |
| GPI-1485 + Remdesivir (3 | >90 | >90 | — |
| μM) | | | |
| Molnupiravir (single conc.) | >0.3 | >0.3 | — |
| Remdesivir (single conc.) | >0.3 | >0.3 | — |

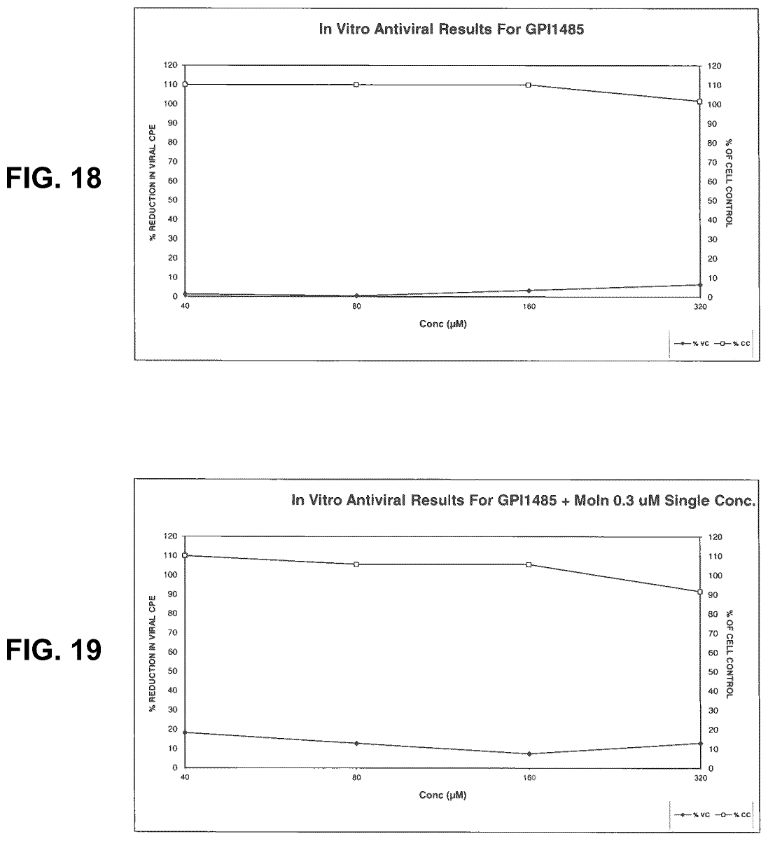

Reduction in viral CPE (solid circles) and cell control (open circles) for GPI-1485 alone, as a function of GPI-1485 concentration (40-320 μM), are shown graphically in FIG. 18.

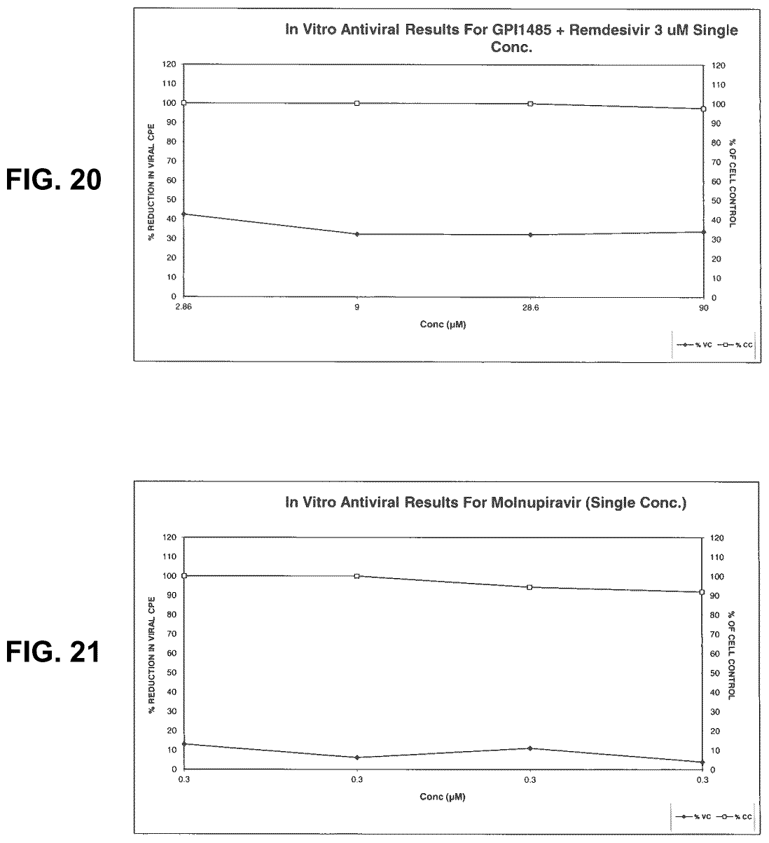

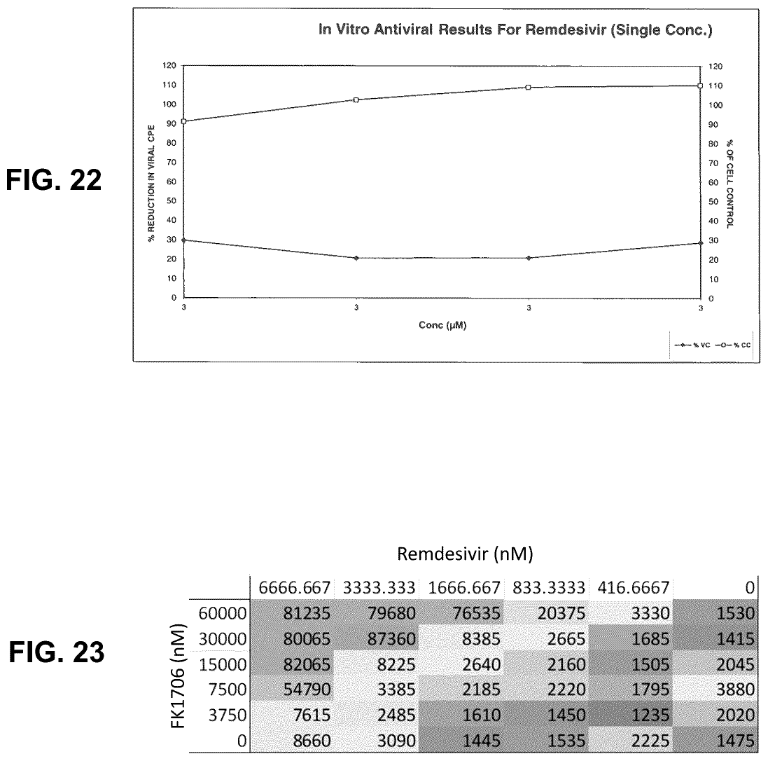

Results from co-administration of GPI-1485 and sub-therapeutic molnupiravir (0.3 μM) are shown graphically in FIG. 19, and the results from co-administration of GPI-1485 and sub-therapeutic remdesivir (3 μM) are shown graphically in FIG. 20. Antiviral activity of sub-therapeutic molnupiravir (0.3 μM) monotherapy is shown in FIG. 21 for comparison, and for sub-therapeutic remdesivir (3 μM) monotherapy is shown in FIG. 22 for comparison. Alone, GPI-1485 showed little reduction in SARS COV-2 virus (0-6%) across the range of 40-320 μM. Molnupiravir administered alone at sub-therapeutic levels (e.g., 0.3 μM) showed very little reduction in SARS COV-2 virus (4-13%); sub-therapeutic (3 μM) remdesivir also showed very little reduction in SARS COV-2 virus (20-30%). GPI-1485 (40-320 μM) showed little additive effects when co-administered with 0.3 μM molnupiravir (7-18%) or 3 UM remdesivir (32-42%).

These data demonstrate that GPI-1485 combined with sub-therapeutic molnupiravir or sub-therapeutic remdesivir does not show synergistic suppression of the SARS-COV-2 virus.

### Example 10

The synergistic therapeutic effects of FK1706 and remdesivir or molnupiravir were determined in a live SARS-COV-2 virus assay measuring the ability of compounds to reverse the viral induced cytopathic effect (CPE) in Vero E6 host cells. Viral infection and replication leads to a loss of host cell viability, which is indirectly measured by an endpoint assessment of host cell viability after 72 hr. The CPE reduction assay indirectly monitors the ability of compounds to inhibit viral replication and infection through various molecular mechanisms, including direct inhibition of viral entry or enzymatic processes as well as acting on host pathways that modulate viral replication. Compounds with antiviral activity protect the host cells from the CPE of the virus, thereby increasing viability.

### Protocol:

All compounds were dissolved in DMSO. To prepare assay ready plates (ARPs), compounds were acoustically dispensed as 45 nl per compound per well (90 nl/well total DMSO volume) in 6×6 matrix combinations into 384-well assay plates (Greiner, black clear bottom, tissue culture treated plates). Each compound was titrated at 5-point 1:2 titrations with the sixth point being DMSO only (to test single agent dose response). Each matrix block was tested as n=3.

### CPE Assay Protocol

Host cells were Vero E6 cells selected for high ACE2 expression (Severson W E, et al., J Biomol Screen. vol. 12(1), pages 33-40 (February 2007)). Cells were premixed with SARS-CoV-2 for 5-10 min, then dispensed into assay ready plates (pre-dispensed with compounds and controls). Cells and virus were incubated with compounds for 72 hr, then viability was assayed by Vero E6 host cell ATP content. This assay can tolerate 90 nl of DMSO without loss of signal.
1. Assay ready plates were thawed at room temperature.
2. 5 μl/well of media was dispensed into assay plates (MEM, 1% Pen/Strep/GlutaMax, 1% HEPES, 2% HI FBS).
3. Dispensed 25 μl/well of Vero E6 cells inoculated with SARS COV-2 (USA_WA1/2020) at multiplicity of infection (MOI) of 0.002 suspended in media in columns 3-24. Final cell density was 4000 cells/well.
4. Dispensed 25 μl/well of Vero E6 cells in media in columns 1-2. Final cell density was 4000 cells/well.
5. Assay plates were incubated for 72 hr at 37° C., 5% CO₂, 90% humidity.
6. Dispensed 30 μl/well of CellTiter-Glo (Promega, Cat #G7573).
7. Plates were incubated for 10 min at room temperature.
8. Luminescence signal was read on Perkin Elmer Envision or BMG CLARIOstar plate reader.

### Controls and Normalization (on Every Plate):

Columns 1-2: No virus control (Vero E6+DMSO). Normalized to 100% CPE rescue.
Columns 23-24, rows 1-12: DMSO control (Vero E6+SARS-COV-2+DMSO). Normalized to 0% CPE rescue.
Columns 23-24, rows 13-16: 60 nL of 2 mg/ml Calpain inhibitor IV. These wells serve as the compound positive control, but are not used for normalizations. Signal should be the same as no virus controls (columns 1-2).

### Cytotoxicity Assay Protocol

This is a cell-based assay measuring host cell ATP content as a readout for cytotoxicity in Vero E6 cells selected for high ACE2 expression (Severson W E, et al., J Biomol Screen. vol. 12(1), pages 33-40 (February 2007)). This assay is used as a counter screen assay to the SARS-COV-2 CPE Assay.
1. Assay ready plates were thawed at room temperature.
2. 5 μl/well of media was dispensed into assay plates (MEM, 1% Pen/Strep/GlutaMax, 1% HEPES, 2% HI FBS).
3. Vero E6 in media (MEM, 1% Pen/Strep/GlutaMax, 1% HEPES, 2% HI FBS) was dispensed 25 μl/well. The final cell density was 4000 cells/well.
4. Assay plates were incubated for 72 hr at 37 C, 5% CO₂, 90% humidity.
5. 30 μl/well of CellTiter-Glo (Promega #G7573) was dispensed into the assay plate.
6. Plates were incubated for 10 min at room temperature.
7. Luminescence signal was read on Perkin Elmer Envision or BMG CLARIOstar plate reader.

### Controls and Normalization (on Every Plate):

Columns 1-2: DMSO control (Vero E6+DMSO). Normalized as 100% viability.
Columns 23-24: Negative control (Vero E6+Hyamine). Normalized as 0% viability.

### Assay Results

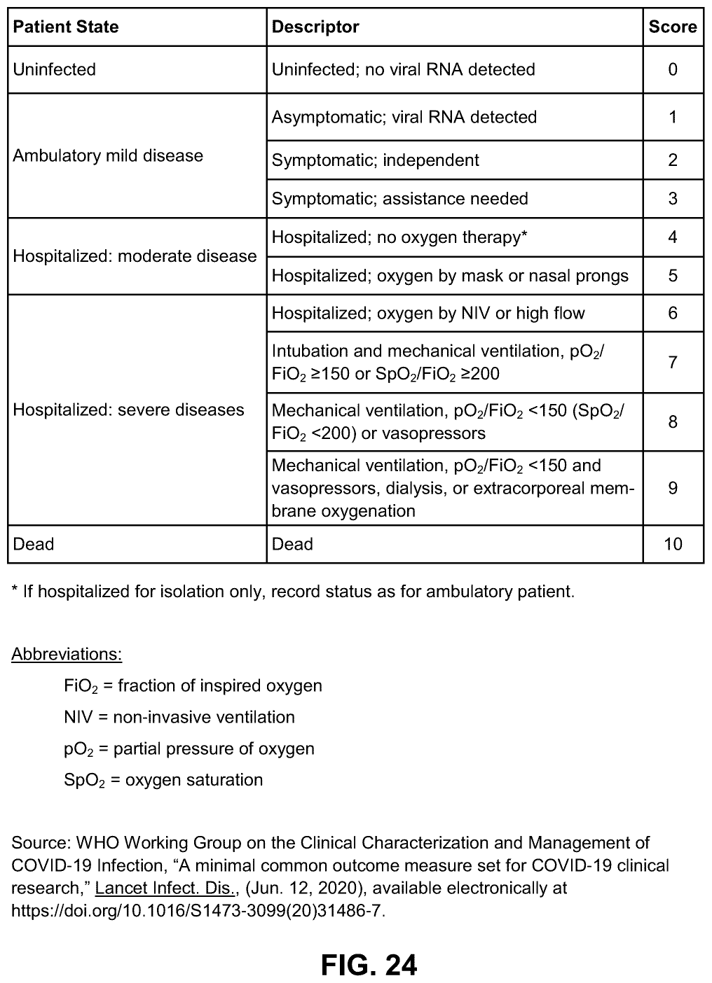

Results are summarized in Table 5 and FIGS. 23-29.

Briefly, none of the three compounds showed activity in either the CPE assay or the cytotoxicity assay as single agents at the concentrations tested. However, the combination of remdesivir and FK1706 showed rescue of CPE without overt cytotoxicity.

Remdesivir alone at concentrations up to 6,666.667 nM and FK1706 alone at concentrations up to 60,000 nM showed no significant antiviral activity in the assay. When remdesivir and FK1706 were combined, synergistic activity (solid border and bold text in Table 5 marked region “(A)”; green cells in FIG. 23) inhibiting SARS-COV-2 and protecting the host cells from the virus cytopathic effect was seen.

### Example 11. Oral Therapy in Non-Hospitalized Patients with Mild or Moderate COVID-19

A Phase 3 multicenter randomized double blind parallel group placebo controlled study will be conducted to evaluate the safety, tolerability and efficacy of an FKBP ligand (e.g., FK1706) administered orally in combination with an oral antiviral protease or polymerase inhibitor (e.g., molnupiravir (MK-4482)). The primary hypothesis is that the FKBP ligand in combination with molnupiravir will be superior to molnupiravir alone as assessed by the percentage of participants who are hospitalized and/or die through Day 29 and/or by any one or more of the following Primary and/or Secondary Outcome Measures.

### Primary Outcome Measures:

1. Percentage of participants who are hospitalized and/or die [Time Frame: Up to 29 days]
   Hospitalization (all cause) is ≥24 hours of acute care in a hospital or similar acute care facility. Death is due to any cause.
2. Percentage of participants with an adverse event (AE)
   An AE is any untoward medical occurrence in a clinical study participant, temporally associated with the use of study intervention, whether or not considered related to the study intervention.
3. Percentage of participants who discontinued study intervention due to an AE
   An AE is any untoward medical occurrence in a clinical study participant, temporally associated with the use of study intervention, whether or not considered related to the study intervention.

### Secondary Outcome Measures:

1. Time to sustained resolution or improvement of each targeted COVID-19 sign/symptom [Time Frame: Up to 29 days]
   The number of days from randomization to the first day on or before study Day 29 for sustained resolution or improvement of each sign/symptom will be assessed.
2. Time to progression of each targeted COVID-19 sign/symptom [Time Frame: Up to 29 days]
   The number of days from randomization to the first day on or before study Day 29 for each targeted self-reported sign/symptom will be assessed.
3. WHO 11-point outcomes score on a scale [Time Frame: Up to 29 days]
   The World Health Organization (WHO) outcome scale is an 11-point ordinal score that categorizes clinical progression. Score ranges from 0 to 10 with higher score indicating clinical progression.
4. Time to Patient Reported Global Daily impression [Time Frame: From Day 0 to Day 28]
   Proportion of patients showing sustained Alleviation of Symptoms by Days 4, 7, 10, 14, 17, 21, 24, and 28, where alleviation is defined as symptom severity of 0 or 1 and is maintained to day 28.
5. Time to improvement in symptoms [Time Frame: Day 0-28]
   Time (number of days) to improvement in each of the symptoms of stuffy or runny nose, sore throat, shortness of breath, cough, lack of energy or tiredness, muscle or body aches, headache, chills or shivering, feeling hot or feverish, nausea, vomiting, diarrhea by at least 1 grade over baseline (Day 1 Self-Assessment Severity).
6. Proportion of patients that progress to severe COVID-19 [Time Frame: Day 28]
   Progression to severe COVID-19 (severe COVID-19 defined as O₂ saturation of <94% at rest, all cause hospitalization, or death)
7. Reduction in Death Related to COVID-19 [Time Frame: Day 21]
   Proportion of patients dying (a) from any cause, or (b) from a COVID-19 associated complication
8. Reduction in Patient Hospitalization [Time Frame: Day 28]
   Proportion of patients hospitalized: (a) from any cause, or (b) from a COVID-19 associated complication
9. Reduction in incidence of hospitalization for respiratory distress or O₂ saturation [Time Frame: Day 28]
   Incidence of hospitalization for respiratory distress or O₂ saturation <93%
10. Time to negative conversion of detectable SARS-COV-2 viral RNA in RT-PCR assays of saliva [Time Frame: Day 0 to Day 10]
   Time (number of days) to negative conversion of detectable SARS-COV-2 viral RNA in RT-PCR assays of saliva, from start of study treatment to study day 10
11. Proportion of patients showing negative conversion of detectable SARS-COV-2 viral RNA in saliva [Time Frame: Days 2, 4, 6, 8 and 10]
   Proportion of patients showing negative conversion of detectable SARS-COV-2 viral RNA in saliva on Study Days 2, 4, 6, 8 and 10
12. Reduction in need for respiratory intervention (i.e., need for mechanical ventilation, ECMO, noninvasive positive pressure ventilation, or high-flow nasal cannula oxygen delivery).

The population for this study will be men and women, 18 years and older with documentation of laboratory confirmed severe acute respiratory syndrome coronavirus 2 (SARS-COV-2) infection with sample collection≤5 days prior to the day of randomization. PCR is the preferred method for confirming infection status. Serological tests that detect host antibodies generated in response to recent or prior infection will not be allowed. Patients in the study will include:
Individuals satisfying the above criteria and who have had initial onset of signs/symptoms attributable to COVID-19 for ≤5 days prior to the day of randomization and at least 1 of the following sign/symptom attributable to COVID-19 on the day of randomization:
   Presents at randomization with mild or moderate COVID-19.
   Presents at randomization with at least 1 characteristic or underlying medical condition associated with an increased risk of severe illness from COVID-19.
Male study subjects must agree to the following during the intervention period and for at least 4 days after the last dose of study intervention: Either abstain from heterosexual intercourse as their preferred and usual lifestyle (abstinent on a long term and persistent basis) and agree to remain abstinent; or must agree to use contraception.
Female study subjects must not be pregnant or breastfeeding at randomization, and at least one of the following conditions must apply: Is not a woman of child bearing potential (WOCBP); or is a WOCBP and using a contraceptive method that is highly effective (a low user dependency method OR a user dependent method in combination with barrier method), or be abstinent from heterosexual intercourse as their preferred and usual lifestyle (abstinent on a long-term and persistent basis) for at least 4 days after the last dose of study intervention; a WOCBP must have a negative highly sensitive pregnancy test (urine or serum test is required) within 24 hours before the first dose of study intervention.

Patients will not be eligible for the study if they present at intake screening or randomization with any of the following:
currently hospitalized or is expected to need hospitalization for COVID-19 within 48 hours of randomization.
on dialysis or has reduced estimated glomerular filtration rate (eGFR)<30. mL/min/1.73 m² by the Modification of Diet in Renal Disease (MDRD) equation.
Have any of the following conditions:
   human immunodeficiency virus (HIV) with a recent viral load >50 copies/mL (regardless of CD4 count) or an AIDS-defining illness in the past 6 months;
   HIV-positive diagnosis with recent viral load ≤50 copies/mL and are not adherent to a stable antiretroviral therapy regimen;
   A neutrophilic granulocyte absolute count <500/mm³;
   Have a history of hepatitis B virus (HBV) or hepatitis C virus (HCV) with cirrhosis, end-stage liver disease, hepatocellular carcinoma, aspartate aminotransferase (AST) and/or alanine aminotransferase (ALT)>3× upper limit of normal at screening;
   Have a platelet count <100,000/μL or received a platelet transfusion in the 5 days prior to randomization;
   Is taking or is anticipated to require any prohibited therapies;
   Is unwilling to abstain from participating in another interventional clinical study through Day 29 with an investigational compound or device, including those for COVID-19 therapeutics;
   Have hypersensitivity or other contraindication to any of the components of the study interventions as determined by the investigator; or
   Have any condition for which, in the opinion of the investigator, participation would not be in the best interest of the participant or that could prevent, limit, or confound the protocol-specified assessments including but not limited to: participants who are not expected to survive longer than 48 hours after randomization, or participants with a recent history of mechanical ventilation, or participants with conditions that could limit gastrointestinal absorption of capsule contents.
Minimal baseline severity score for COVID-19-related symptoms: at least two symptoms with a score of 2 or higher.

### Example 12. FKBP Ligand in Combination with Remdesivir for Hospitalized Patients with Mild/Moderate or Severe SARS-COV-2

A Phase 3 multicenter, randomized, open-label, phase 3 trial involving hospitalized patients with confirmed SARS-COV-2 infection, oxygen saturation of 94% or less while they were breathing ambient air, and radiologic evidence of pneumonia will be conducted. Patients will be randomly assigned in a 1:1 ratio to receive intravenous remdesivir or the combination of remdesivir with an FKBP ligand (e.g. FK1706). The primary hypothesis is that the FKBP ligand in combination with remdesivir will be superior to remdesivir alone as assessed by the percentage of participants who are hospitalized and/or die through Day 29.

Patients in the study will include hospitalized patients at least 12 years of age presenting with:
SARS-COV-2 infection confirmed by polymerase-chain-reaction assay within 4 days before randomization;
Radiographic evidence of pulmonary infiltrates; and
   Mild/moderate disease defined as SpO₂>94% and respiratory rate <24 breaths/minute without supplemental oxygen; or
   Severe disease defined as an SpO₂≤94% on room air, a respiratory rate ≥24 breaths/minute, an oxygen requirement, or a requirement for mechanical ventilation.

Patients excluded from the study will include those presenting at intake screening or randomization with any of the following:
Extracorporeal membrane oxygenation (ECMO) at screening;
Signs of multiorgan failure;
Alanine aminotransferase (ALT) or aspartate aminotransferase (AST) levels greater than times the upper limit of the normal range;
Estimated creatinine clearance of less than 50 ml per minute (by the Cockcroft-Gault formula); or
Receiving concurrent treatment (within 24 hours before the start of trial treatment) with other agents with putative activity against COVID-19.

Patients will be randomly assigned in a 1:1 ratio to receive intravenous treatment with remdesivir alone or remdesivir in combination with an FKBP ligand. All the patients will receive 200 mg of remdesivir on day 1, followed by 100 mg of remdesivir once daily. Patients in the remdesivir monotherapy treatment group may also receive a placebo in place of the FKBP ligand. Both treatment groups will continue supportive therapy at the discretion of the investigator throughout the duration of the trial.

### Clinical and Laboratory Monitoring

Patients will be assessed by physical examination and by documentation of respiratory status, adverse events, and concomitant medications. On trial days 1, 3, 5, 8, 10, and 14, blood samples will be obtained for complete blood count and measurement of creatinine, glucose, total bilirubin, and liver aminotransferases.

The clinical status of patients will be assessed daily on a 7-point ordinal scale (see below) from day 1 through 14 or until discharge. The worst (i.e., the lowest) score from each day will be recorded.

### End Points

The primary efficacy end point will be the clinical status assessed on day 14 on a 7-point ordinal scale consisting of the following categories:
1: death;
2: hospitalized, receiving invasive mechanical ventilation or ECMO;
3: hospitalized, receiving noninvasive ventilation or high-flow oxygen devices;
4: hospitalized, requiring low-flow supplemental oxygen;
5: hospitalized, not requiring supplemental oxygen but receiving ongoing medical care (related or not related to COVID-19);
6: hospitalized, requiring neither supplemental oxygen nor ongoing medical care; and
7: not hospitalized

The secondary end point of the trial will be the proportion of patients with adverse events that occurred on or after the first dose of remdesivir or FKBP ligand for up to 30 days after the last dose.

Prespecified exploratory end points will include:
the time to clinical improvement (defined as an improvement of at least 2 points from baseline on the 7-point ordinal scale),
the time to recovery defined as an improvement from a baseline score of 2 to 5 to a score of 6 or 7,
the time to modified recovery (defined as an improvement from a baseline score of 2 to 4 to a score of 5 to 7 or from a score of 5 to a score of 6 or 7),
death from any cause, and
thromboembolism.

### Statistical Analysis

The prespecified primary analysis, performed after all patients complete 14 days in the trial using the proportional odds model, will include treatment as the independent variable and baseline clinical status as a continuous covariate. The expected conclusion that remdesivir in combination with an FKBP ligand is superior to remdesivir alone will be confirmed if the lower bound of the two-sided 95% confidence interval of the odds ratio on day 14 was greater than 1. The stratified Wilcoxon rank-sum test is prespecified to compare the treatment groups in case the proportional odds assumption was not met. For time-to-event end points (such as the time to clinical improvement, the time to recovery, and the time to modified recovery), the hazard ratio and its 95% confidence interval will be estimated from a cause-specific proportional-hazards model that includes treatment and baseline clinical status as covariates and treated death as the competing risk. For events associated with prespecified times (e.g., days 5, 7, 11, and 14), the difference in the proportion of patients with an event under evaluation (such as clinical improvement, recovery, and modified recovery) between treatment groups and its 95% confidence interval will be estimated from the Mantel-Haenszel proportions, with adjustment according to baseline clinical status.

1: A method of treating or preventing a coronavirus infection in a subject in need thereof, the method comprising co-administering to the subject in need thereof a non-immunosuppressive FKBP ligand or derivative thereof and an antiviral agent.

2: The method of claim 1, wherein the FKBP ligand or derivative thereof is one or more of: 15-O-demethyl tacrolimus, GPI1046, GPI-1485, ElteN378, V10367, L-685818, WAY 124466, AG5473, AG5507, meridamycin, and FK1706.

3: The method of claim 1, wherein the antiviral agent is selected from the group consisting of: remdesivir, molnupiravir, favipiravir (Avigan), AT-527, triazavirin (riamilovir), ribavirin, plitidepsin, danoprevir (Prezcobix), ASC09F (also referred to as TMC-310911), tenofovir, atazanavir, oseltamivir (Tamiflu), emtricitabine, PF-07304814, PF-07321332, S-217622, and daclatasvir.

4: The method of claim 1, wherein the antiviral agent is an RNA polymerase inhibitor.

5: The method of claim 4, wherein the RNA polymerase inhibitor is remdesivir or molnupiravir.

6-8. (canceled)

9: The method of claim 1, wherein the antiviral agent is a protease inhibitor.

10: The method of claim 9, wherein the protease inhibitor is M128533.

11: The method of claim 1, wherein the FKBP ligand or derivative thereof is FK1706 or ElteN378.

12: The method of claim 1, wherein the method is a method of preventing a coronavirus in a subject who does not have a diagnosed coronavirus infection.

13-14. (canceled)

15: The method of claim 1, wherein an immune response associated with the subject is not substantially suppressed after the step of co-administering the FKBP ligand or derivative thereof and the antiviral agent to the subject.

16: The method of claim 1, wherein the coronavirus infection is a SARS COV-2 viral infection.

17: The method of claim 1, wherein the FKBP ligand is administered to the subject at a sub-therapeutic dose.

18: A pharmaceutical composition comprising:
an FKBP ligand or a derivative thereof; and
an antiviral agent,
wherein administration of the pharmaceutical composition to a subject does not substantially suppress an immune response of the subject.

19: The pharmaceutical composition of claim 18, wherein the FKBP ligand or derivative thereof is one or more of: 15-O-demethyl tacrolimus, GPI1046, GPI-1485, ElteN378, V10367, L-685818, WAY 124466, AG5473, AG5507, meridamycin, and FK1706.

20: The pharmaceutical composition of claim 18, wherein the antiviral agent is selected from the group consisting of: remdesivir, molnupiravir, favipiravir (Avigan), AT-527, triazavirin (riamilovir), ribavirin, plitidepsin, danoprevir (Prezcobix), ASC09F (also referred to as TMC-310911), tenofovir, atazanavir, oseltamivir (Tamiflu), emtricitabine, PF-07304814, PF-07321332, S-217622, and daclatasvir.

21: The pharmaceutical composition of claim 18, wherein the antiviral agent is an RNA polymerase inhibitor.

22: The pharmaceutical composition of claim 18, wherein the antiviral agent is present in a sub-therapeutic amount.

23: The pharmaceutical composition of claim 21, wherein the RNA polymerase inhibitor is remdesivir or molnupiravir.

24-25. (canceled)

26: The pharmaceutical composition of claim 18, wherein the antiviral agent is a protease inhibitor.

27: The pharmaceutical composition of claim 26, wherein the protease inhibitor is M128533.

28-29. (canceled)
